(19) United States
(12) Patent Application Publication
(10) Pub. No.: US 2006/0257956 A1
(43) Pub. Date: Nov. 16, 2006

(54) Method and device for chemical or biological analysis by a sensor with a monolithic chamber in the form of a multi-microtubular sheaf and a lateral integration measuring transducer

(76) Inventors: Frederic Basset, Londres (FR); Jean-Marie Billiotte, Londres (FR); Petr Ivanovich Nikitin, Moscow (RU)

Correspondence Address: YOUNG & THOMPSON, 745 SOUTH 23RD STREET, 2ND FLOOR, ARLINGTON, VA, 22202, US

(21) Appl. No.: 10/563,055

(22) PCT Filed: Jul. 1, 2004
(86) PCT No.: PCT/FR04/01707

§ 371 Date: Jun. 1, 2006

(30) Foreign Application Priority Data
Jul. 4, 2003 (FR) 03/08227

(51) Int. Cl. G01N 33/554; G06F 19/00

(52) U.S. Cl. 435/7.32; 702/19

(57) ABSTRACT

A method and device for chemical or biological analysis by a sensor provided with a monolithic chamber in the form of a multi-microtubular sheaf and a lateral integration measuring transducer. The method improves the usual method for a sensor evaluation of an analyte concentration in a fluid sample by multi-canalising in parallel the fluid sample fraction through channels of a monolithic reaction chamber, positioning a transducer system fully outside the surface of the shell of the reaction chamber and strictly in front of the lateral face thereof, carrying out an integral measurement of signals generated by the analyte combination and a receiver, which is also arranged in the reaction chamber, by a lateral transducer system coevally in all channels of the reaction chamber such that the presence of the analyte in all channels of the reaction chamber is simultaneously and globally quantified.

### TECHNICAL FIELD OF THE INVENTION

The invention relates to the technical field of chemical and/or biological chemical and/or biological sensors. The object of a sensor is to carry out a method of evaluation of the concentration of an analyte in a fluid sample. The analyte elements are generally -soluble chemical entities or live or dead micro-organisms, or parts of micro-organisms (enzyme, antibody, antigen, microbial cell, gas, ion, metabolite, micro-organism, protein, oligonucleotide . . . ). The analyte can be found in any fluid sample such as a liquid or a gas (air . . . ). The object of a sensor is to convert the concentration of the analyte included in the fluid sample into an exploitable analytical (generally electrical) signal.

Amongst the different types of analytical systems, a sensor is understood to mean a device for measurement of concentration which combines:
within a reaction chamber, a chemical compound, known as a receptor, for (molecular) recognition of the analyte and emission (possibly with the aid of another compound known as an indicator which may be merged with the receptor) of an elementary physico-chemical recognition signal,
and a hardware system, known as a transducer, for reception of this signal.

This distinguishes a sensor of an analytical system which incorporates either other supplementary separation systems, such as high-pressure liquid chromatography (HPLC), or additional hardware equipment, as is the case for flux injection analysis (FIA) devices.

Inside a sensor, the receptor is a chemical and/or biological compound, which is both:
adapted to recognise the analyte,
and capable of generating, in combination with the analyte (and possibly an indicator), an elementary signal to measure the presence of an analyte element.

The transducer is a physical means (hardware) which converts the action of the receptor (or bioreceptor)
leading to the generation of a multitude of events of recognition of analyte elements,
into a global signal which makes it possible to quantify the presence of this analyte element in the sample.

The sensors can be classified by means of the following parameters which determine their capacities:
the type of receptor used,
the way in which the analyte interfaces with the receptor,
the type of elementary signal emitted indicating the presence of an analyte element,
the geometry of the reaction chamber,
the structure and the geometry of the transduction means and the position thereof with respect to the reaction chamber, that is to say the relative geometry of the reaction chamber/transducer pairing.

The invention relates specifically to the technical field of (chemical and/or biological) sensor methods and devices
of which the reaction chamber is monolithic and multi-microtubular,
and of which the transducer is entirely situated outside the test volume of the reaction chamber.

The invention relates to a method for improving the performance of chemical and/or biological sensors as well as a novel sensor geometry for carrying out this improvement.

### PRIOR ART

The principle of sensors is widely exploited by the prior art. A particular category of sensors known in the prior art is constituted by biosensors. In a biosensor the system of chemical recognition uses a biochemical mechanism. In this case the receptor can be an antibody, an enzyme, a cell, a portion of cell membrane or of organelle, a fraction of cell tissue or an organism . . . .

By way of example, let us consider the classical operating principle of a biosensor for the measurement of glucose in a liquid, using as receptor (bioreceptor) a glucose oxidase enzyme in such a way as to implement the following catalysis reaction:
glucose+O₂→gluconic acid+H₂O₂

This type of sensor with enzymatic receptor was described for the first time by Clark and Lyons in 1962.

The transduction, that is to say the measurement of glucose present in the liquid, can theoretically be carried out:
by an oxygen transducer which measures the ratio between the oxygen present before and after the enzymatic recognition reaction,
by a pH transducer which measures the production gluconic acid in the course of the enzymatic recognition reaction,
or by a peroxide transducer which measures the production of H₂O₂ in the course of the enzymatic recognition reaction.

It will be noted that according to these three methods the transducer is situated in part upstream and downstream of the reaction chamber.

In order to permit a sensor to effect a rapid and precise analyte concentration measurement it will be understood intuitively that it is appropriate:
on the one hand that the recognition step should be the most “bijective”, that is to say that the greatest proportion possible of analyte elements should be recognised by a receptor element, (it is rare that a plurality of identical receptor elements identify the same analyte element),
and on the other hand that the transduction should be the most sensitive possible and in order to do this that it should concern the greatest possible quantity of analyte elements recognised.

If, furthermore, consideration is given to sensors of the solid phase type, that is to say of which at least one of the elements (analyte or receptor or indicator) is immobilised on the test surface of the reaction chamber (and all parameters also being imposed), the chemistry requires that the “bijectivity” of the recognition is all the more great:
since the “surface” for exchange between analyte elements and receptor elements within the reaction chamber is great,
and since the “average test distance” between the fluid sample and the test surface is small.

We shall call
the average test distance: the average of the distances between the elementary portions of fluid sample inside the test chamber of the sensor and the test surface,
and the average test cross-section of the reaction chamber: twice the average test distance.

Particularly in the case of sensors of the solid phase type and for chemical reasons, therefore, it is appropriate in so far as is possible to produce a reaction chamber having a test volume:
with a large test surface,
and with a small average test section,
whilst having a reduced global test volume in order to limit the space required and the consumption of fluid sample and of reagents.

That is to say that it is preferable to optimise the reaction parameters of the reaction chamber chemically in such a way
that the ratio “per unit area” [between the test surface of the reaction chamber and its average test cross-section] is high,
and that the “sensitivity” ratio [between the test surface of the reaction chamber and its test volume] is high.

Moreover, for reasons of sensitivity of measurement, it is desirable to physically effect the measurement of transduction over the greatest number of recognition events possible. It will therefore be appreciated that these efficiency parameters of the sensors are a priori contradictory.

The prior art seeks to achieve these objectives in several directions.

A first direction of the prior art, with a “bidimensional” reaction chamber, aims to carry out the identification by the receptor of the analyte within a fine (quasi-planar) bidimensional test volume. This bidimensional category comprises first of all devices for testing on a capillary membrane. A test on a capillary membrane is understood to mean a method of analysis carried out within a fine membrane consisting of a porous medium such as blotting paper. A specific indicator of the analyte sought is immobilised on a specific test zone of the membrane. After deposition on the membrane, the liquid sample including the analyte travels through the porous zone by capillarity. When the liquid sample reaches the specific test zone, the analyte combines with the indicator. This reaction involves a chemiluminescent phenomenon such as fluorescence or colouration of the specific test zone. This enables a conclusion to be drawn in a binary manner as to the presence or absence of analyte.

This “per unit area” strategy of testing on a capillary membrane satisfies the required “chemical” conditions described above, that is to say:
large test surface,
small average test cross-section,
small global volume.

However, it will be noted that these tests on a capillary membrane do not constitute sensors in the precise sense above. They do not have any physical transducer system. Therefore they are a technological background for sensors with multitubular reaction chambers. The result is generally read off directly by eye. The consequence is that these tests on a capillary membrane are above all qualitative (binary). They do not allow precise quantification of the concentration of analyte. Moreover, the “bidimensional” membrane geometry, that is to say in a thin layer, has the drawback that it is not very sensitive to the presence of analyte (small volume). The visual phenomenon which appears is the result of the physico-chemical effects on a surface layer. As a result these membrane tests can only be used for substantial concentrations of analyte, (typically 10⁶/ml for micro-organisms).

In order to overcome this obstacle of low sensitivity, use is sometimes made of an initial phase of enrichment of the sample. Typically in microbiology a culture of the starting sample can be made in a Petri dish in order to multiply the number of micro-organisms present considerably before analysis. The detrimental consequence of this amplification phase, which currently lasts for 24 to 72 hours, is a characteristic slowness, regretted by all users, which is costly and inconvenient.

A second direction of the prior art, with a “tridimensional” reaction chamber, aims to carry out the identification by the receptor of the analyte within a tridimensional test volume.

A first sub-variant of this second sensor strategy can be described as sensors with a chamber “with a low ratio of surface area to volume”.

An example of this strategy is the use, as analysis cell, of a hollow conical support pipette in the VIDAS devices of the company BioMerieux (France). The fluid sample is taken inside a conical support coated internally with a receptor. Then reagents and washing solutions are successively drawn in and forced into the interior cone of the pipette. The pairs (receptor-analyte) are detached from the surface of the cone and forced into a well where they are counted by spectrophotometry. This method does indeed make it possible to automate a large number of tests and to facilitate the manipulation by the operator of the samples and the reagents. However, although it is much used in serology, it does not make it possible in microbiology to dispense with the preliminary growth phases. It will be understood that one of the shortcomings of this type of sensor is that the ratio “per unit area” [between the test surface of the reaction chamber and its average test cross-section] is low. The consequence is that the instantaneous probability of capture of an analyte element by a receptor element is low. As a result the global quantity of elementary signals of recognition of analyte elements is low. The resulting signal picked up by the transducer is not very sensitive. It necessitates a period of incubation for amplification which considerably lengthens the time taken for carrying out a test. Typically a test by a device of this type necessitates a period of preparation of 18 hours followed by a process of measurement lasting 15 to 45 minutes.

A second sub-variant of this second strategy may be described as “multi-tridimensional”. Typically reaction chambers are used with a capillary test volume.

It is known in the prior art to use capillary structures in the field of sensors. The person skilled in the art knows well the porous structures obtained in particular by assembling microspheres of polyethylene or of polystyrene or cellulose derivative fibres, agglutinated to form a porous network. These capillary structures are intended to immobilise analytes, and to be traversed by reagents. The tests on a membrane described above use these techniques. This is the material forming the basis of the pregnancy tests currently used at home or the tests for detection of streptococci in cases of angina. As has been seen above, the reading of these tests is purely visual based on the appearance of a coloration. A major drawback of this type of test is its limited sensitivity [the “useful volume” of colouration being limited to the surface part of the porous layer] which limits the use thereof at high concentrations of analytes. Moreover, it has been seen that these systems do not constitute sensors because they do not have a transducer. This is therefore a technological background which is very far from the invention.

Also known in the prior art are the manufacture and the use of monolithic multitubular structures for applications not associated with analysis. The (US) company Schott produces and markets them for applications in laboratories and in optoelectronics. The (US) company Burle produces and markets them for applications in electronic tubes and photomultipliers. The (US) company Collimated Holes produces and markets them for applications associated with optical fibres. Nowadays these chambers with a multi-microtubular structure according to the prior art typically have microtube diameters of five microns to one millimetre. The geometry of the microtubes is generally of a circular, hexagonal or square cross-section. The number of microtubes assembled is approximately 200,000. The global cross-section of the chamber is of the order of 25 mm. They are conventionally made from glass (borosilicate or lead silicate). The usual application according to the prior art of these multi-microtubular structures concerns: the collimation of fluxes of gas and of X-rays, the calibration of a leakage, the monitoring of air flows, use as a differential pressure barrier, filtration, optoelectronics, the input window of lasers. An advanced application proposed by Burle is the realisation of a generator of a stream of electrons amplified when a potential difference is applied to the two ends of the chamber with multi-microtubular structure. Therefore these are not applications of sensors.

It is likewise known in the prior art to bring together a parallel bundle of tubes with membrane walls spaced from one another in order to produce a dialysis system. The structure is not monolithic. The tubes, spaced from one another, are connected in parallel at a first end to a blood inlet connector and at a second end to a blood outlet connector. The whole assembly is placed inside a sheath through which the dialysate circulates. This system does not use a chemical receptor within the tubes, nor a transducer. Therefore this is not an application of sensors.

The document WO 02/094440 A2 (“Microchip integrated multichannel electroosmotic pumping system”) describes another application of a monolithic array of capillary tubes where this array constitutes an electro-osmotic pump for use in chips or in micro-machines. However, this device does not comprise a transducer. Moreover it is not an application of a sensor. This is a technological background very distant from the invention. The sensors of the prior art according to the second sub-variant of this second “multi-tridimensional” strategy aim to carry out the identification by the receptor of the analyte within a multi-channel tridimensional test volume. This strategy may be described as “with a low ratio of surface area to volume”. However, it will be seen below that in this case, the prior art is not concerned with the structure of the transducer and the geometry of the multi-tubular chamber/transducer pair.

The technological background of the invention includes the manufacture and the use of non-monolithic multi-tubular “multi-tridimensional” structures for applications associated with analysis.

A non-monolithic analysis sensor with a multi-tubular chamber is described in the patent U.S. Pat. No. 6,517,778 (“Immunoassays in capillary tubes”). The test volume is constituted by one single capillary tube or a small number of capillary tubes which are separate from one another. The fluid sample is placed in one or several wells of a receiving tray which can be disposed of after use. It is mixed there with a reagent, drawn into one or several of the capillary test tubes, separate within a cartridge, connected to an analysis device. The analyte elements react with receptor elements borne by the surface of the capillary test tube(s). The test tubes are then washed in order to stop the reaction and dried. Each capillary test tube is then exposed to a lamp in order to create a fluorescence signal which is detected by a transducer. It should be noted that:
on the one hand, each capillary tube is separate and distant from the others (this is a non-monolithic structure),
and on the other hand, the transduction measurement is carried out tube by tube (it does not describe a transducer geometry laterally encompassing all of the tubes).

It will be understood that a first drawback of this type of sensor is that its “sensitivity” ratio [between the internal test surface of the reaction chamber and its test volume] is low because it uses a small number of tubes spaced from one another. As a result its sensitivity is low. A second drawback of this device is that its cartridge for the tubes is bulky, awkward and difficult to transport and to manipulate (because of its size).

The prior art includes the manufacture and the use of monolithic multi-tubular structures for applications associated with analysis. The use of these structures in chemical analysis has been considerably accelerated by the disclosure (particularly in the field of pharmaceutical research) of so-called high-throughput screening techniques. These make use of “libraries” of different reagents simultaneously implemented on plates bearing typically 96 reaction wells.

The patent U.S. Pat. No. 6,027,873 (“Multithrough hole testing plate for high throughput screening”) describes a screening device which uses a multi-microtubular structure in order to link the reservoirs of a library of products at the bottom of the wells of a multi-well plate. The proximal ends (on the side of the multi-well plate) are welded to one another to constitute a monolithic multi-tubular reaction head. The distal ends (on the side of the reservoirs of the library) remain separate in the form of flexible tubes. The object of this is to circumvent the difficulty of filling wells of very small size. This is not an application of a sensor. This document does not describe any reaction of the analyte receptor type within the tubes. Moreover, no measurement transducer for detection of analyte is described. Quite clearly this document is not concerned with the geometry of the transducer/reaction chamber pair.

The document US 2002/164824 (“Method and apparatus based on bundled capillaries for high throughput screening”) also essentially describes a high-throughput screening device for chemical compounds of the general type described above and not a sensor. Nevertheless this document may be considered as forming part of technological background of the invention because the use of these capillary structures has been proposed briefly as a possible support for immunological tests (Claims 55 et seq). In the description of this device by way of a sensor of the immunological competitive type, it is equipped with hollow optical fibres which concentrically line the interior of each microtube. These optical fibres constitute a bundle of transducers. According to the technique proposed, the transduction is therefore effected separately inside each of the tubes of the structure. The signal is picked up at the end of each of the tubes. The principal drawback of a sensor with a transducer bundle of this type would be the complexity of the geometry of the transducer/microchannel pairs and the associated manufacturing costs. Furthermore, any branching of the multitude of optical fibres would render the assembly awkward and fragile. This would render the manufacture of disposable mobile test cartridges according to this technique prohibitive.

The document WO 02/10761 A1 (“Microarrays and their manufacture by slicing”) likewise describes the manufacture of a high-throughput screening device” where each tube or cylinder of an array of tubes or of cylinders is coated with a different biological agent. These arrays are sectioned, perpendicular to their principal direction, in order to constitute slices through which a sample is passed. However, the colouration of each tube is observed at one of its ends. There is no transducer placed laterally with respect to the array of tubes. Nor is there any integration into one signal of the signals of the plurality of tubes, since a signal is observed for each tube.

The document US 2002/086325 A1 (“Affinity detecting/analytical chip, method for production thereof, detection method and detection system using same”) describes an array of tubes made from glass with a resin support moulded on it. The tubes are coated internally with molecules capable of different specific binding reactions. A sample is passed through the array and certain components of the sample can be retained specifically by the molecules fixed to the interior of the tubes. By the application of a luminous flux to one end of the tubes, the colouration is then observed at the other end. This varies according to whether or not certain components of the sample have been retained inside the tubes. There is no transducer placed laterally with respect to the array of tubes, and there is no integration into one signal of the signals of the plurality of tubes, since a signal is observed for each tube.

The patent U.S. Pat. No. 5,690,894 (“High density array fabrication and readout method for a fiber optic biosensor”) describes the manufacture and the use of biosensors comprising a plurality of optical fibres, each optical fibre having specific elements of an analyte attached to its sensitive end. Each optical fibre acts purely as a transducer and ensures the sole transport of an item of optical information to the other end. The information of a fibre is either displayed by an operator or processed by a digital device. This patent does not describe multi-channelling of the sample through a multitubular reaction chamber. Therefore this is technological background which is very far from the invention.

A third direction of the prior art of sensors is concerned more particularly with the aspect of sensitivity of measurement as referred to above.

The patent EP 1,262,766 (“Method for analyzing a mixture of biological and/or chemical components using magnetic particles and device for the implementation of said method”) teaches the use of porous capillary structures as reaction support within the test volume in order to increase the “sensitivity” ratio [between the interior test surface of the reaction chamber and its test volume] and therefore the density of recognition events in the interior of the test volume. The sensor uses antibodies as receptor elements and super-paramagnetic particles as indicator elements. The transduction is based on the application of a magnetic field to the test volume and the measurement of the magnetic induction which results from the magnetisation of all the indicator elements present in the test volume. The only manner described for creating a porous capillary structure is based on an assembly of polyethylene micro-granules. Therefore this document is not concerned with the specific field of sensors with a multi-tubular reaction chamber. Nor does this document describe the relative geometry of the reaction chamber/transducer pair. The principal drawback of the type of porous capillary structure envisaged by micro-granules is that it creates a test volume with multiple random cavities in respect of which it could be noted that it causes numerous false detection events particularly due to granules trapped in the cavities. This type of sensor is imprecise. Another drawback of this type of porous capillary structure is its “sensitivity” ratio [between the test surface of the reaction chamber and its test volume]. It is lower than that of a multi-tubular structure such that its sensitivity is lower.

Finally, amongst the technological background of the invention mention may be made of different systems for chemical treatment using structures which are multi-tubular but not intended for the production of sensors. Thus the patent U.S. Pat. No. 6,027,627 “Automated parallel capillary electrophoretic system” describes an automated electrophoretic system. The device uses a cartridge which comprises:
a plurality-of capillary tubes joined at their ends, but not touching over their length,
and an equal plurality of parallel electrophoresis tubes.

At a first end the capillary tubes are joined to microtitre trays, and at the other end to electrophoresis tubes. The device comprises a system for supplying gel which serves as a medium for migration. This makes it possible to effect the capillary electrophoresis of samples present in each of the wells of the tray. The micro-tubular structure (both of the capillary tubes and of the electrophoresis tubes) is not monolithic. Furthermore this system does not carry out any reaction of recognition of analyte by a receptor. Finally, the system does not include a transducer. This is not an application of a sensor.

### SUMMARY OF THE INVENTION

In its most general form, the invention relates to a method of evaluation of the concentration of analyte elements of an analyte present in a fluid sample. The expression “analyte elements” is used to mean soluble chemical entities or live or dead micro-organisms, or parts of micro-organisms. The invention relates to an improvement in the usual method of operation of a sensor. “Sensor” is used to mean a device for evaluation of the concentration of analyte elements of an analyte present in a fluid sample constituted by
a reaction chamber which has in its interior a test volume within which is channelled the fraction of the fluid sample to be analysed,
and a measurement transducer system.

The test volume is circumscribed by an enclosing reaction surface. Topologically the enclosing reaction surface is defined as the smallest continuous surface surrounding the said test volume. Conventionally this enclosing surface is constituted by
a permeable upstream front face,
a permeable downstream front face situated opposite the downstream permeable face,
and a substantially cylindrical impermeable lateral face connected by its two ends to the peripheries of the two upstream and downstream faces.

Any sensor utilises an active component (chemical and/or biological) known as a receptor which is placed in contact with the fluid sample within the test volume. The receptor elements have an affinity with the analyte elements in order to detect them. The receptor also has the property [alone or in combination with another active component known as an indicator likewise introduced into the test volume] of modifying by an elementary signal a measurable extensive state variable (physical and/or chemical), at each occurrence [or according to a certain law of probability], at the time of an event of recognition of an analyte element by a receptor element.

The transducer system for measurement of the extensive state variable is a hardware component which makes it possible to quantify the presence of the analyte elements in the fluid sample.

The method of evaluation of the concentration according to the invention is characterised in that in combination:
on the one hand, the fraction of the fluid sample is multi-channelled in parallel, through a sensor provided with a monolithic reaction chamber in the form of a multi-microtubular array,
on the other hand, the lateral transducer system for integral measurement of the extensive state variable is positioned entirely outside the test surface of the reaction chamber, and strictly facing the impermeable lateral face,
and finally with the aid of the lateral transducer system for integral measurement of the extensive state variable an integral measurement of the variations of the said extensive state variable is carried out simultaneously in all the channels of the reaction chamber.

More precisely, a reaction chamber is used which is constituted by the joining of a plurality of multi-tangent cylindrical micro-tubular channels in such a way as to delimit a dense plurality of separate convex elementary volumes, disposed in array, open at their two ends, and of which the joining constitutes a convex global test volume.
The channels are cylindrical in the sense that they each delimit an internal elementary surface generated topologically by the displacement, along an elementary central line of a continuous virtual skeleton, of a curve of closed and continuous shape placed substantially perpendicularly.
The channels have lengths [that is to say the lengths of the elementary central lines] which are substantially equal.
The channels are micro-tubular, that is to say that they have an elementary internal cross-section perpendicular to the elementary central line which has at least a selective transverse dimension several orders of magnitude smaller than its length (typically of the order of 1000 times smaller).
The channels are disposed substantially parallel, that is to say that their elementary central lines are disposed substantially parallel.
The channels are multi-tangent. That is to say that each micro-tube is in longitudinal contact over substantially all of its length with at least one other adjacent micro-tube. As a result the assembly of micro-tubular channels constitutes a dense monolithic array.

The non-convex global test volume is circumscribed by the enclosing reaction surface of which the permeable upstream and downstream front faces are situated at right angles to the inlet and outlet cross-sections of the micro-tubular channels.

Finally, a lateral integral measurement transducer system is implemented which effects an integral measurement ΔE=Σ_{k=1 . . . n} Σᵢⱼ (dE)ᵢⱼₖ, (that is to say a summation) of the variations of the said extensive state variable simultaneously for all the elementary volumes at once, and for all the elementary signals (dE)ᵢⱼₖ in each elementary tube at once, through the impermeable lateral face. As a result the presence of the analyte elements in the fluid sample is quantified globally in all the micro-tubular channels of the reaction chamber at the same time.

### LIST OF DRAWINGS

FIGS. 1, 1a and 1b show the operational principles of the method of evaluation of the concentration of analytes according to the invention with the aid of a cartridge-type sensor in the form of a monolithic multi-tubular array and a lateral integral transducer.

FIGS. 2 and 3a to 3d describe the dimensional relations and geometric structures of arrays of microtubular channels recommended by the invention in order to produce a test cartridge.

FIGS. 4a to 4d show the different stages of movement of fluids and reagents through the reaction chamber during the operation of an immunological sensor having an antibody-type receptor and an indicator with super-paramagnetic micro-granules according to the invention.

FIGS. 5a and 5b show in perspective and in cross-section a first preferred embodiment according to the invention of a disposable mobile cylindrical cartridge for a sensor.

FIG. 6a shows in perspective a second preferred embodiment according to the invention of a disposable mobile conical cartridge for a sensor.

FIG. 6b shows a preferred embodiment of the conical cartridge.

FIGS. 7a and 7b show in perspective two preferred variants of a third embodiment according to the invention of a disposable mobile cartridge with a monolithic chamber in the form of a mono-periodic lamellar multi-tubular array.

FIG. 8 describes schematically the method of operation according to the invention of a multi-location sensor (in two parts).

FIGS. 9a, 9d and 9e describe schematically an embodiment according to the invention of a multi-location sensor including a sampling gun and a disclosing/measurement device.

FIGS. 9b and 9c describe an embodiment of needle cartridges according to the invention.

FIG. 10 describes in greater detail the functional diagram of the disclosing/measurement device of FIG. 9e.

FIG. 11 describes a variant of a test cartridge extended by a sampling cone.

FIGS. 12a, 12b, 13a to 13d and 14 describe another variant of a linear robot device according to the invention.

FIGS. 15, 15a and 15b describe a robot variant of a multi-location sensor with a carousel according to the invention.

FIGS. 16a and 16b describe the operational principle of a sequential multi-location robot device for analysis according to the invention.

FIGS. 17a and 17b describe a variant of a multi-chamber test cartridge.

FIG. 17c describes a multi-chamber test multi-cartridge.

FIGS. 18a and 18b describe a simplified variant of a sampling syringe according to the invention.

FIGS. 19a to 19d describe schematically four embodiments of a multi-location sensor according to the invention.

FIGS. 20a to 20c describe in a simplified manner in perspective, schematically and in cross-section, a magnetic transducer device according to the invention.

FIG. 21 describes a multi-analyte sensor produced according to the invention.

FIGS. 22, 22a and 22b describe a preferred method according to invention for producing the network in the form of a multi-tubular array of a sensor cartridge.

FIGS. 23a to 23c illustrate the sequence of the reactions of an analysis of the sandwich type.

FIGS. 24a and 24b illustrate the sequence the reactions of an displacement analysis.

FIGS. 25a and 25b illustrate the sequence of the reactions of an analysis by replacement.

### DETAILED DESCRIPTION OF THE INVENTION

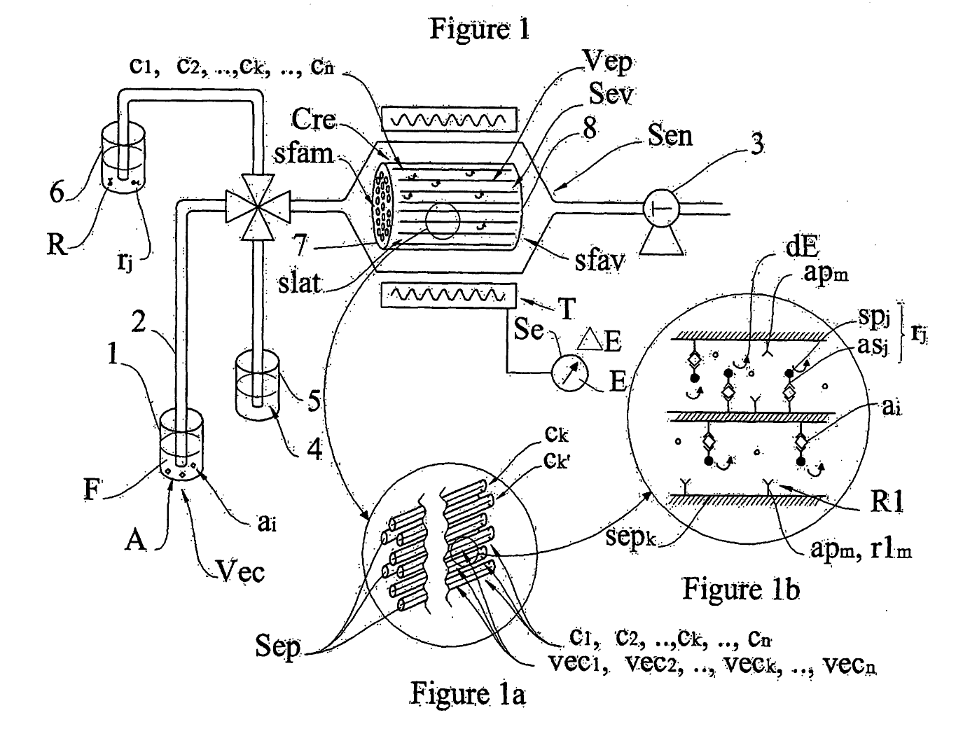

FIG. 1 describes using a particular example the method according to the invention for operation of a sensor (Sen) for the evaluation of the concentration of analyte elements (aᵢ) of an analyte (A) which are present in a fluid sample (F), initially contained in a sample volume (Vec). As is the case with any chemical or biological sensor, the operation of the sensor (Sen) comprises the following steps:
a fraction of the fluid sample (F) is channelled within a test volume (Vep),
the fluid sample (F) is placed in contact, within the test volume (Vep), with an active component (chemical and/or biological) known as a receptor (R),
by means of a measurement transducer system (T) the presence of the analyte elements (aᵢ) in the fluid sample (F) is measured.

The test volume (Vep) is circumscribed by an enclosing reaction surface (Sev). The enclosing reaction surface (Sev) is defined as the smallest continuous surface surrounding the said test volume (Vep). The sensor (Sen) is principally constituted by a reaction chamber (Cre) which forms the interior of the enclosing reaction surface (Sev). The enclosing reaction surface (Sev) has a permeable upstream front face (sfam), a permeable downstream front face (sfav) (situated opposite the permeable upstream face (sfam)), and face lateral substantially cylindrical impermeable face (slat). The lateral face (slat) is connected by its two ends to the peripheries (7, 8) of the two upstream (sfam) and downstream (sfav) front faces.

By way of example, the sensor (Sen) according to the invention described in FIGS. 1, 1a and 1b is of the immunomagnetic type. Its purpose is to evaluate by an analysis of the sandwich type the presence of analyte elements (aᵢ) constituted by bacteria of the genus Cryptosporidium present in a fluid sample (F) of drinking water. The receptor elements (r1ₘ) of a first active receptor component (R1) (chemical and/or biological), constituted in this instance by primary antibodies (apₘ) [specific to the genus Cryptosporidium], are fixed on the test surface (Sep). They have an affinity with the analyte elements (aᵢ) in order to detect them and to immobilise them while they are being multi-channelled through the reaction chamber (Cre). The active receptor component (R) is present in a beaker (6). The receptor elements (rⱼ) have the property of modifying a measurable extensive state variable (physical and/or chemical) (E) by an elementary signal (dE) at each occurrence [or according to a certain law of probability], during an event of recognition of an analyte element (aᵢ) by a receptor element (rⱼ). In this particular case the receptor elements (rⱼ) are constituted by pairs of a secondary antibody (asⱼ) [specific to the genus Cryptosporidium] onto which is grafted a super-paramagnetic micro-granule (spⱼ). The super-paramagnetic micro-granules (spⱼ) are devoid of magnetic activity in the absence of an external field, but induce a disturbance of an external magnetic field when it is applied thereto.

The measurement transducer (T) serves to measure the variations of the said extensive state variable (E), in this case the magnetic field, in such a way as to quantify the presence of the analyte elements (aᵢ) in the fluid sample (F) in the form of an exploitable analytical signal (Se). In this case the transducer (T) measures the disturbances generated by the super-magnetic micro-granules (spⱼ) upon application of a magnetic field (H) with regard to the impermeable lateral surface (slat).

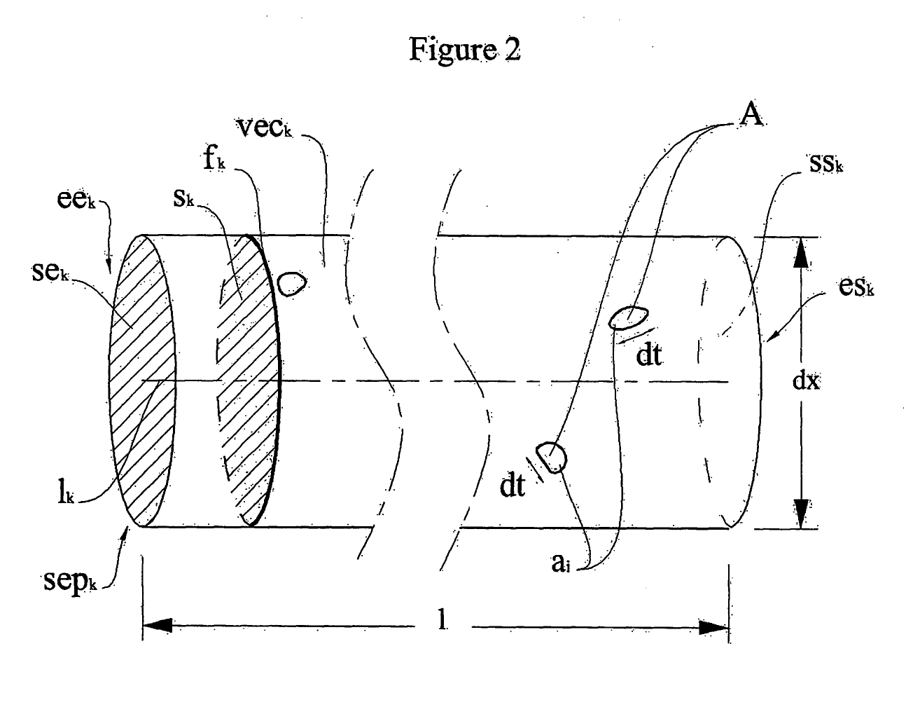

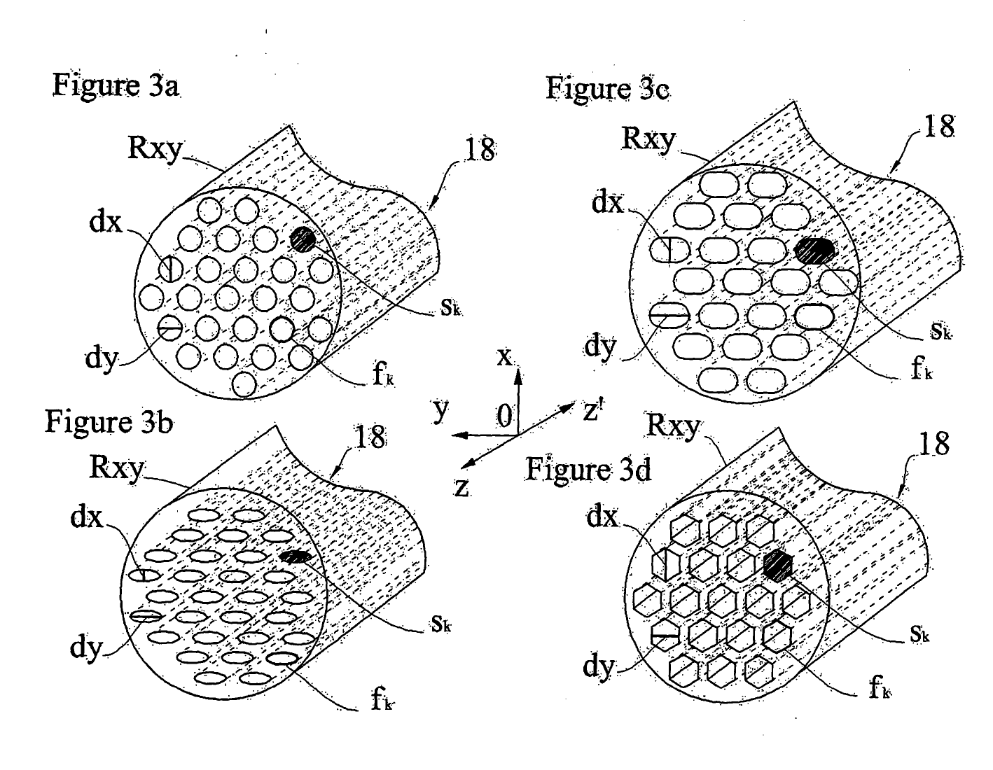

However, according to the method according to the invention it will be noted that the fraction of the fluid sample (F), [bacteria-laden water], is multi-channelled in parallel through a reaction chamber (Cre). The reaction chamber (Cre) is monolithic and multi-tubular, constituted by the joining of a plurality of multi-tangent cylindrical micro-tubular channels (c₁, c₂, . . . , cₖ, . . . , cₙ) into an array. FIGS. 1a and 2 describe in greater detail the configuration of the micro-tubular channels (cₖ) inside the reaction chamber (Cre). The channels (cₖ) are cylindrical, that is to say that they each delimit an elementary interior surface (sepₖ) generated topologically by the displacement along an elementary central line of a continuous virtual skeleton (lₖ) of a curve of continuous and closed shape (fₖ) placed substantially perpendicularly. The channels (cₖ) can have a curve (fₖ) of circular, elliptical, oval or polygonal shape as illustrated by FIGS. 3a to 3d. The channels (cₖ) have substantially equal lengths (l), that is to say that the lengths of their elementary central lines (lₖ) are equal. The channels (cₖ) are microtubular, that is to say that their elementary internal cross-section (sₖ) perpendicular to the elementary central line (lₖ) has at least a selective transverse dimension (dx) several orders of magnitude smaller than their length (l) (typically of the order of 1000 times smaller). The channels (cₖ) are disposed substantially parallel in an array, that is to say that their elementary central lines (lₖ) are disposed substantially parallel. Moreover, they are multi-tangent. That is to say that each micro-tube (cₖ) is in longitudinal contact over substantially all of its length with at least one other adjacent micro-tube (c_{k′}). In this way the reaction chamber (Cre) delimits internally a dense plurality of separate adjacent convex elementary volumes (vec₁, vec₂, . . . , vecₖ, . . . , vecₙ) which are open at their two ends (eeₖ, esₖ). Joining thereof forms a non-convex global test volume (Vep). The non-convex global test volume (Vep) is circumscribed by the enclosing reaction surface (Sev) of which the permeable upstream (sfam) and downstream (sfav) front faces are situated at right angles to the inlet (seₖ) and outlet (ssₖ) cross-sections of the micro-tubular channels (c₁, c₂, . . . , cₖ, . . . , cₙ).

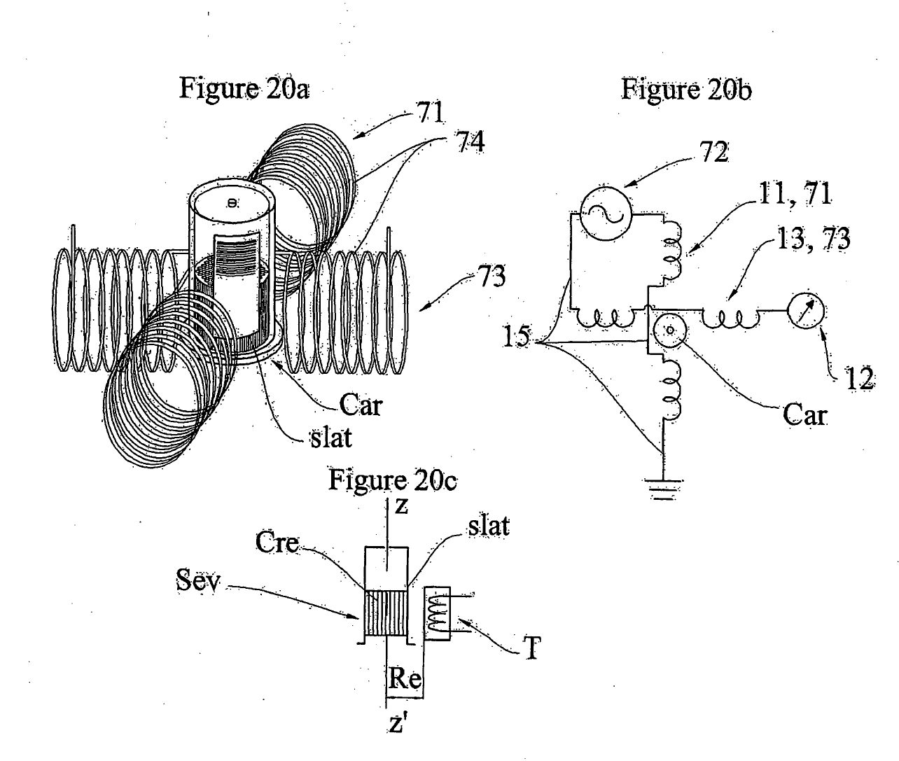

In addition to a reaction chamber (Cre) in the form of a multi-tubular array, the sensor (Sen) is provided with a lateral magnetic transducer system for integral measurement (T) of the extensive state variable (E). This is shown in greater detail in FIGS. 20a to 20c. It is constituted by an electromagnetic field emitter (11) formed by a primary winding (71) connected to a primary current source (72) and a magnetic field receiver (13) formed by a secondary winding (73) connected to a secondary current analysis device (12). The primary (71) and secondary (73) windings (74) surround the impermeable lateral face (slat) of the reaction chamber (Cre) in the form of a multi-microtubular array. It will be noted that the active part of the lateral magnetic transducer for integral measurement (T), and in particular the primary (71) and secondary (73) windings thereof, is situated entirely outside the enclosing surface (Sev) of the reaction chamber (Cre) and strictly facing the impermeable lateral face (slat).

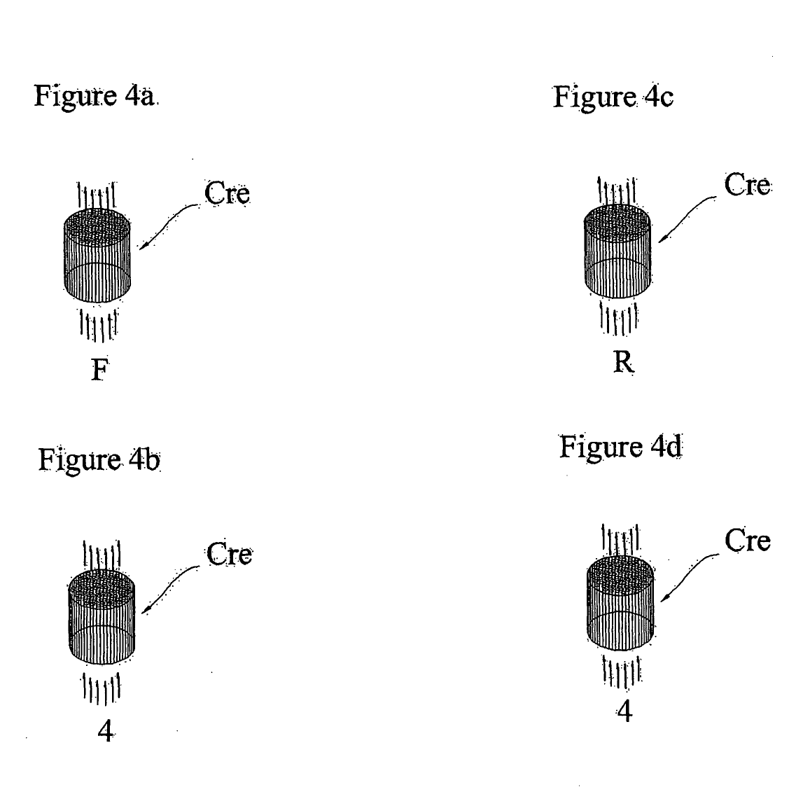

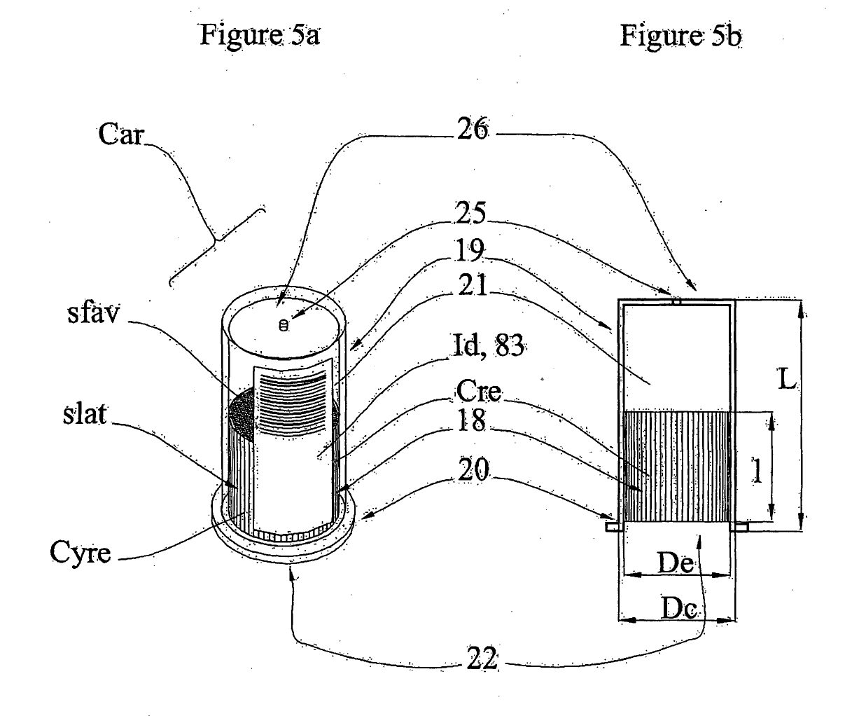

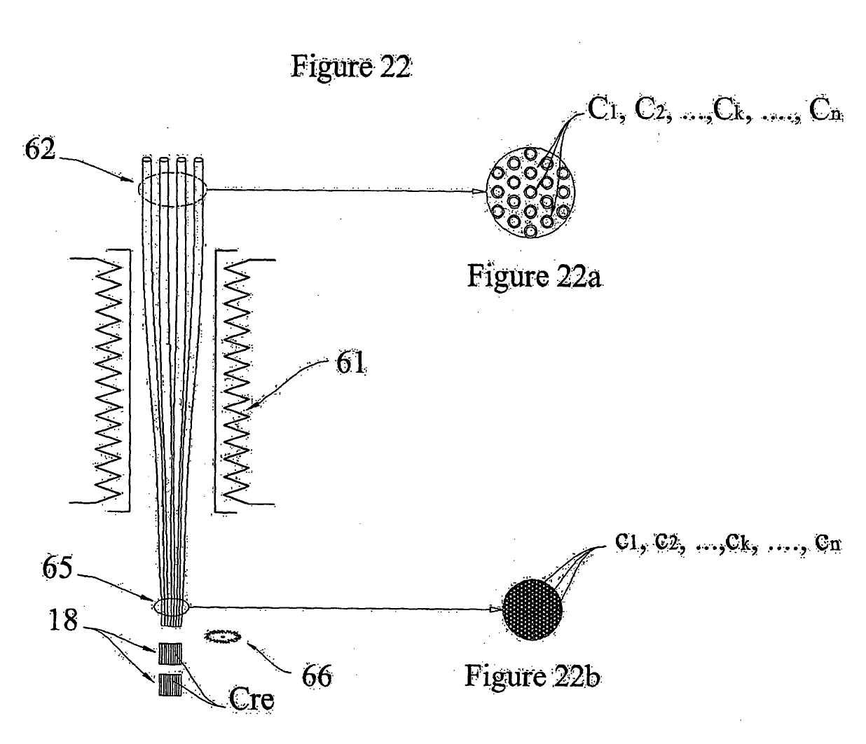

As is apparent from FIG. 1, the sensor (Sen) functions in the following manner. The fluid sample (F) is situated initially in the sample volume (Vec) of a beaker (1). It is removed by means of a suction tube (2) immersed in the beaker (1) and is drawn in by means of a dosaging pump (3) situated downstream. It is multi-channelled through the micro-tubular channels (cₖ) of the test cartridge (Car) which will be described in greater detail in FIGS. 5a and 5b. FIG. 4a describes the suction and the initial multi-channelling of the fluid sample (F) through the reaction chamber (Cre). Then, as is apparent from FIGS. 4b to 4d, the following washing solutions and reagent are multi-channelled successively by suction [and possibly forcing back in certain embodiments] through the micro-tubular channels (cₖ) of the test cartridge (Car). In FIG. 4b, a washing solution (4) comprising a buffer [at pH 7.0] contained in a beaker (5) is drawn in and multi-channelled. Then in FIG. 4c, a receptor suspension (R) contained in the beaker (6) is drawn in and multi-channelled. Finally, in FIG. 4d the washing solution (4) is drawn in again and multi-channelled. The biochemical reactions in this particular case are illustrated in FIGS. 1b and 23a to 23c. The receptor (R1), constituted in this case by antibodies specific to Cryptosporidium, so-called primary antibodies (apₘ), is grafted on the glass wall (sepₖ) of the micro-tubular channels (cₖ) previously activated by silanisation, according to the rules of the art. As the fluid sample (F) passes through, the Cryptosporidium bacteria (aᵢ), if there are any, are specifically retained by these antibodies (apₘ). During the passage of the receptor (R) in excess, the pairs [secondary antibodies (asⱼ)=super-magnetic micro-granules(spⱼ)] are fixed specifically on the immobilised bacteria (aᵢ). The passage of the washing solution (4) makes it possible to eliminate the pairs (asⱼ=spⱼ) not linked to the analyte elements (aᵢ) or linked non-selectively by weaker bonds. Each immobilised bacterium (aᵢ) is then signalled bijectively by a super-paramagnetic micro-granule (spⱼ).

The magnetic lateral integral measurement transducer system (T) preferably functions according to the principle described in the patent EP 1,262,766. A variable magnetic field (H) is applied by means of the winding primary (71) situated on either side of the impermeable lateral surface (slat) of the reaction chamber (Cre). Each super-paramagnetic particle (spⱼ), which is inactive in the absence of an external magnetic field, induces an elementary disturbance (dE)ᵢⱼₖ of the field. By means of the magnetic lateral integral measurement transducer system (T) an integral measurement ΔE=Σ_{k-1 . . . n} Σᵢⱼ (dE)ᵢⱼₖ, (that is to say a summation) of the variations of the said extensive state variable (E) [the magnetic field (H)], is carried out simultaneously for all the elementary volumes (vecₖ) at once, and for all the elementary signals (dE)ᵢⱼₖ in each elementary tube (cₖ) at the same time, through the impermeable lateral face (slat). Then the sum ΔE of these disturbances is measured by means of the secondary winding (73) connected to the secondary current analysis device (12). In this way the presence of the analyte elements (aᵢ) in the fluid sample (F) is quantified globally in all the micro-tubular channels (cₖ) at the same time by means of the field disturbances caused by the micro-granules (spⱼ).

As is apparent in FIG. 2, the invention recommends particular dimensional relations between the analyte elements (aᵢ) and the micro-tubular channels (c₁, c₂, . . . , cₖ, . . . , cₙ). The example described relates to the case where it is desired to evaluate the concentration of analyte elements (aᵢ) of a biological analyte (A) [in this case microscopic fungi or bacteria]. The typical diameter (dt) is typically situated between 0.01 microns and 10 microns. It is recommended by the invention that the monolithic multi-tubular reaction chamber (Cre) is constituted by the reunion of an array of micro-tubular channels (c₁, c₂, . . . , cₖ, . . . , cₙ) of which the said selective transverse dimension (dx) is chosen in correlation with the typical diameter (dt) of the biological analyte elements (aᵢ). Typically the elementary internal cross-section (sₖ) of the micro-tubular channels (cₖ) is chosen in such a way that the said transverse dimensions (dx) are substantially equal to approximately 10 times the typical diameter (dt) of the biological analyte elements (aᵢ), that is to say in particular of the order of magnitude of 10 microns if the biological analyte elements (aᵢ) are bacteria.

The FIGS. 3a to 3d describe the dimensions and geometric relations recommended by the invention for the reaction chamber (Cre) and the transducer (T). Preferably the monolithic bi-periodic multi-microtubular reaction chamber (Cre) is constituted by the joining of a plurality of n (n≅approximately 300 000) micro-tubular channels (c₁, c₂, . . . , cₖ, . . . , cₙ). The micro-tubular channels (cₖ) advantageously have a quasi-revolutionary cross-section, that is to say a cross-section with a curve of continuous shape (fₖ) such as a circle, ellipse, polygon, oval, . . . of which each pair of two perpendicular transverse dimensions (dx, dy) are of the same order of magnitude (d) (d=dx=dy≅of the order of 10 microns). The micro-tubular channels (cₖ) are disposed parallel, adjacent and joined in the form of an array (18) in a common axial direction (zz′) of orientation of their elementary central lines (lₖ). They form a bidimensional periodic network (Rxy) perpendicular to the said common axial direction (zz′) of orientation. As is apparent in FIG. 20c, the lateral integral measurement transducer system (T) of the extensive state variable (E) substantially surrounds the exterior of the impermeable lateral face (slat), at a radial distance (Re) of the order of 7 mm. In the general case and for a cylindrical cartridge (Car), the transducer system is at a distance (Re) of the order of magnitude of Re≅(2.1*√(n/π)*d) of the axis constituted by the said common direction (zz′) of orientation of the reaction chamber (Cre) [that is to say Re≅7 mm for an array (18) of 300 000 micro-tubular channels (c₁, c₂, . . . , cₖ, . . . , cₙ) of internal diameter (d) of 10 microns]. The combination between a cylindrical reaction chamber (Cre) and a lateral integral measurement transducer system (T), substantially surrounding the exterior of the impermeable lateral face (slat) in an annular manner, makes it possible to optimise the ratio of efficiency of measurement (ref=n/Re) between the number n of micro-tubular channels and the distance (Re) between the said lateral integral measurement transducer system (T) and the axis (zz′) of the reaction chamber (Cre).

FIGS. 5a and 5b show in perspective and in cross-section a first preferred embodiment according to the invention of a disposable mobile cylindrical test cartridge (Car) for a sensor (Sen). According to the invention, it is recommended to produce cartridges (Car) constituted by n=300 000 microtubular channels (c₁, c₂, . . . , cₖ, . . . , cₙ) made from glass of 10 microns internal diameter (d). The real diameter of the tubes (cₖ), including the wall, is approximately 1.5 times the internal diameter (d), that is to say 15 microns. In this case the monolithic chamber (Cre) has a diameter (De) of approximately [3*√(n/π)*d], that is to say approximately 10 mm. The reaction chamber (Cre) is surrounded by an equally cylindrical casing (19) of plastics material moulded on it. The casing has a wall with a thickness of approximately 1 mm. As a result the diameter (Dc) of the test cartridge (Car) is 12 mm approximately. Its recommended length (L) is 18 mm approximately. This casing (19) serves for protection and maintenance thereof and facilitates manipulation thereof. The reaction chamber (Cre) is situated at the base of the casing (19) itself with a length (L) which is longer than that (l) of the chamber (Cre). As a result a reservoir (21) is provided in the interior of the casing (19) downstream of the reaction chamber (Cre). The casing (19) is applied forcibly onto the lateral face (slat) of the chamber (Cre) and it is equipped with a lateral sealing element, in this case an annular sealing tongue (20) moulded on at right angles to the upstream face (22) of the base of the test cartridge (Car). This ensures lateral sealing allowing the application of a pressure difference (ΔP) between the upstream (22) and downstream (26) end face of the chamber of the test cartridge (Car) in order to force the flow of the fluid sample (F) through and to protect the cartridge (Car) against lateral leakages and external pollution. An air hole (25) is provided on the downstream end face (26) of the cartridge (Car). The test cartridge is single-use. It can either be thrown away after use or archived for monitoring purposes.

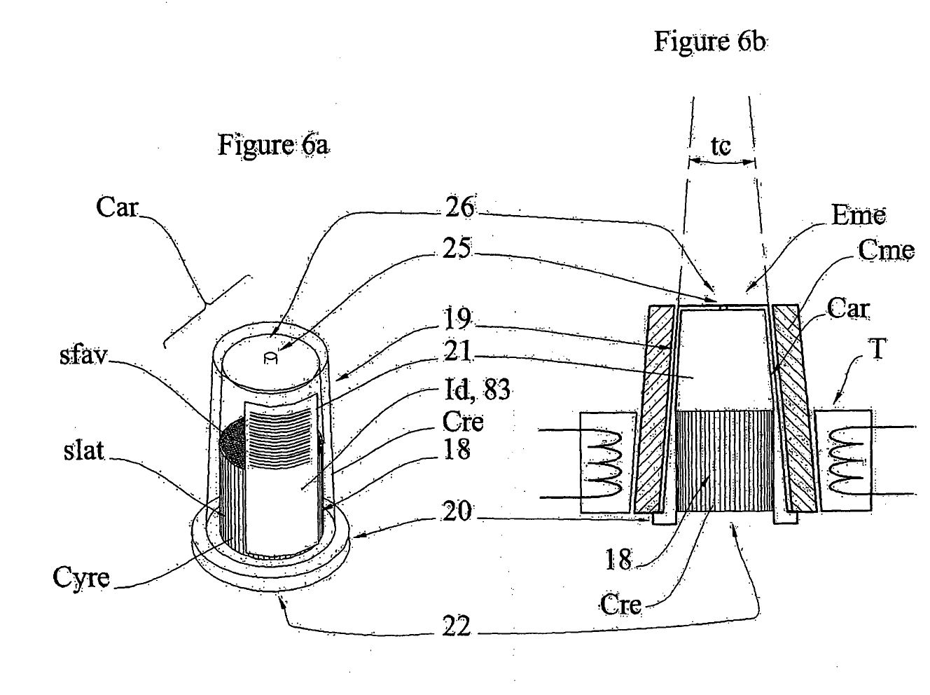

FIGS. 6a and 6b show in perspective and in section a second preferred embodiment according to the invention of a disposable mobile conical test cartridge (Car) for a sensor (Sen). This is similar to the cylindrical test cartridge (Car) described in FIG. 5a and 5b. The reaction chamber (Cre) thereof also has the shape of a quasi-cylinder (Cyre). The difference resides in the fact that the casing (19) moulded on the reaction chamber (Cre) has a conical shape. The use of this test cartridge (Car) of slightly truncated conical shape with an angle (tc) at the top is shown schematically in FIG. 6b. The measurement block (Cme) is given a slightly truncated conical shape, with a top angle (tc). The internal cylindrical measurement cavity (Eme) also has a slightly truncated conical shape with an angle (tc) at the top. The truncated conical test cartridge (Car) is positioned inside the internal truncated conical measurement cavity (Eme) of the measurement block (Cme). This makes it possible to ensure close contact and a reduction in the distance between the lateral integral measurement transducer system (T) and the reaction chamber (Cre). This also permits a possible pressurisation of the micro-tubular channels without leakage between the cartridge (Car) and the measurement block (Cme).

FIGS. 22, 22a and 22b describes a preferred embodiment of the invention for production of the multi-tubular network which constitutes the reaction chamber (Cre) of a cartridge (Car). Beforehand, a large number of glass tubes (C₁, C₂, . . . , Cₖ, . . . , Cₙ) are brought close together and disposed substantially parallel and are introduced into a treatment furnace (61) so as to soften them. Their speed at the outlet of the furnace (62), the so-called drawing speed (Ve), is greater than the feeding speed (Va), they are drawn and in this way a bundle is created in the form of a monolithic continuous array (65) of micro-tubular channels (c₁, c₂, . . . , cₖ, . . . , cₙ). Then this bundle is periodically divided so as to constitute a plurality of monolithic reaction chambers (Cre) in the form of a multi-microtubular array (18).

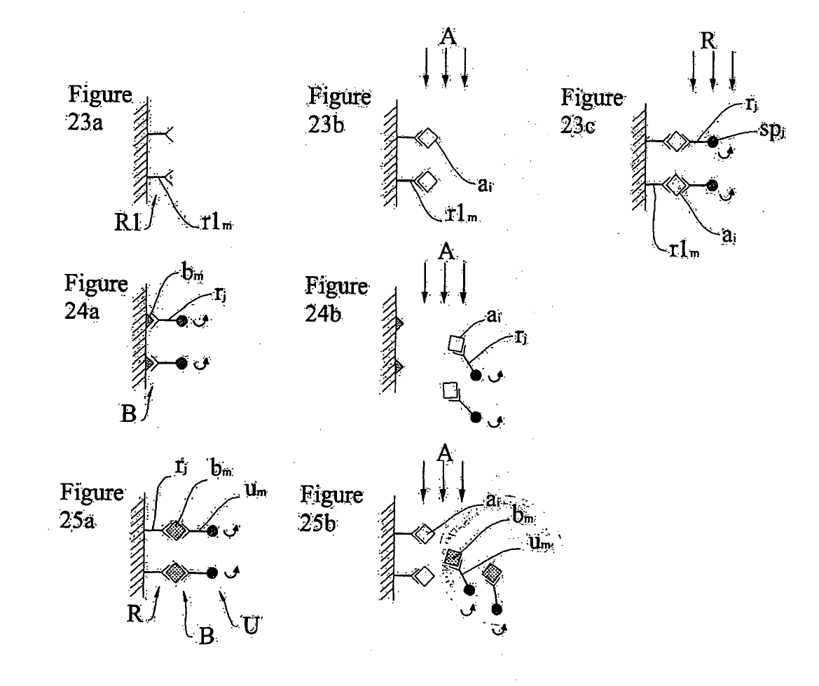

Then, each monolithic reaction chamber (Cre) is conditioned chemically following the rules of the art according to the type of analysis which is to be carried out afterwards. For example, for an analysis of the sandwich type a plurality of receptor elements (r1ₘ) of the receptor component (R1) (for example an antibody or a nucleic acid) which has an affinity for the analyte component (A) are deposited and fixed homogeneously on the internal surface of the plurality of micro-tubular channels (c₁, c₂, . . . , cₖ, . . . , cₙ). The use of the reaction chambers (Cre) thus prepared is described above. All of these steps are described schematically in FIGS. 23a to 23c. For a sensor with displacement analysis, a plurality of analogue elements (bₘ) of an analogue component (B) of the analyte component (A) are deposited and fixed homogeneously on the elementary internal surfaces (sepₖ) of the plurality of micro-tubular channels (c₁, c₂, . . . , cₖ, . . . , cₙ). Then a plurality of receptor elements (rⱼ) of a receptor component (R) are multi-channelled and fixed by affinity for the analogue component (B). The receptor component (R) likewise has an affinity for detecting and fixing the analyte component (A). The principle of the use of reaction chambers (Cre) thus prepared is well known to the person skilled in the art. During the multi-channelling of the analyte component (A), the analyte elements competitively bond with the analogue elements (bₘ) and displace some of the receptor elements (rⱼ) immobilised on the interior surfaces (sepₖ) of the reaction chamber (Cre). By means of the transducer the quantity of receptor elements (rⱼ) within the test volume (Vep) is decreased. All of these steps are described schematically in FIGS. 24a and 24b. For a sensor with analysis by replacement, a plurality of receptor elements (rⱼ) of a receptor component (R) which has an affinity for the analyte component (A) are deposited and fixed homogeneously on the elementary internal surfaces (sepₖ) of the plurality of micro-tubular channels (c₁, c₂, . . . , cₖ, . . . , cₙ). Then an excess of a plurality of analogue elements (bₘ) of the analogue component (B) which likewise has an affinity for the receptor (R) are multi-channelled and fixed. The indicator elements (uₘ) of an indicator component (U) are complexed with the said analogue elements (bₘ). The principle of the use of the reaction chambers (Cre) thus prepared is well known to the person skilled in the art. During the multi-channelling of the fluid sample (F), the analyte elements (aᵢ) competitively bond with the analogue elements (bₘ), take the place of some of the analogue elements (bₘ) and their conjugated indicator elements (uₘ), and are immobilised on the internal surfaces (sepₖ) of the reaction chamber (Cre). By means of the transducer the quantity of indicator elements (uₘ) within the test volume (Vep) is decreased. All of these steps are described schematically in FIGS. 25a and 25b.

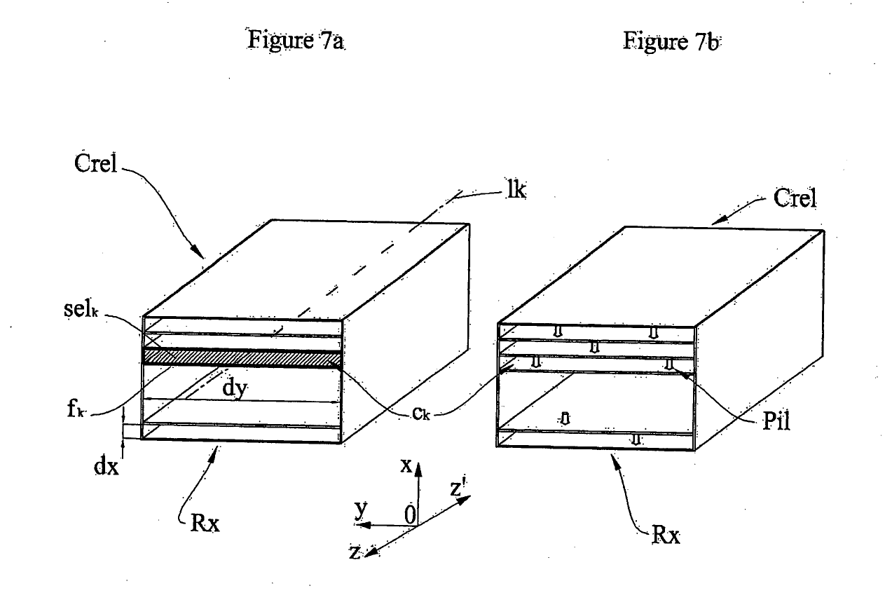

FIGS. 7a and 7b show another preferred embodiment of the invention of a mono-periodic lamellar chamber, according to which the fraction of the fluid sample (F) charged with analyte elements (aᵢ) are multi-channelled in parallel through a monolithic mono-periodic lamellar multi-tubular reaction chamber (Crel). This latter is constituted by the joining of a plurality of n (n≅approximately 1 000) micro-tubular channels (c₁, c₂, . . . , cₖ, . . . , cₙ) of lamellar cross-section (selₖ). The cross-section thereof with a curve (fₖ) is substantially rectangular, and two perpendicular transverse dimensions (dx, dy) are at least of a different order of magnitude (dx<<dy). Typically a selective transverse dimension (dx) is of the order of 10 microns, and the other lateral transverse dimension (dy) is of the order de 10 mm. The microtubular channels (c₁, c₂, . . . , cₖ, . . . , cₙ) of lamellar cross-section are disposed in parallel layers, adjacent and joined in a common planar direction (yOz) of orientation of their elementary central lines (lₖ). The entire assembly constitutes a monodimensional periodic network (Rx) perpendicular to the said common planar direction (yOz) of orientation. FIG. 7b shows a variant of the production of the lamellar reaction chamber of which moreover the structure is reinforced by transverse pillars (Pil).

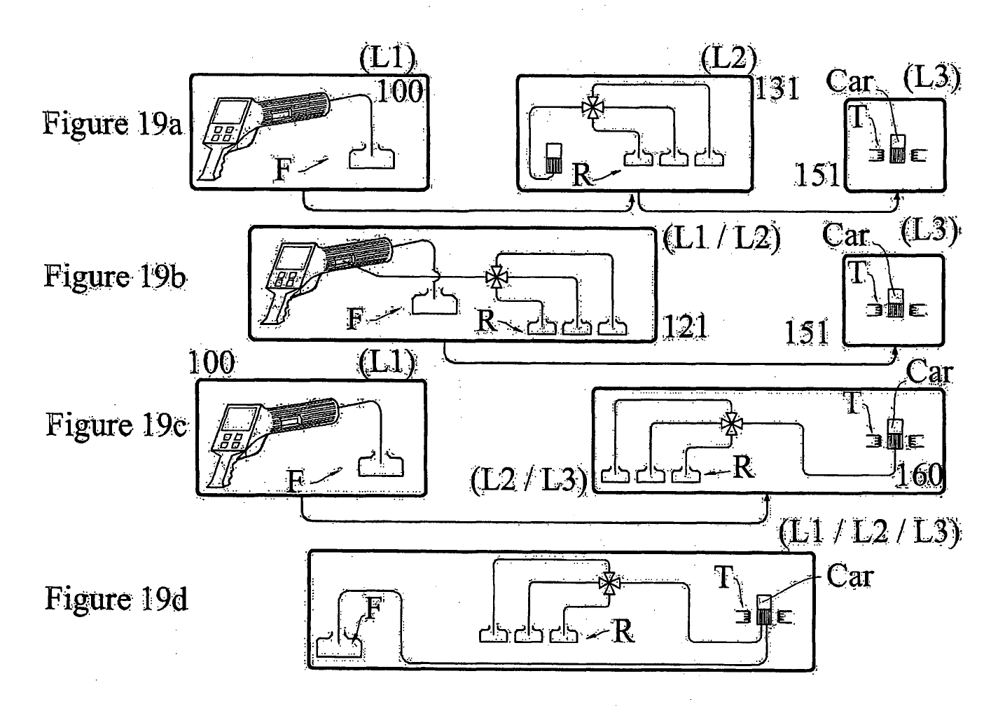

FIGS. 19a to 19d describe four possible diagrams for carrying out the method according to the invention in a multi-location form, according to which the sampling site (L1), indication site (L2) and measurement site (L3) may or may not be separate. In FIG. 19a, the three aforementioned sites are separate. In the first sampling site (L1) the fluid sample (F) is taken by multi-channelling through the test cartridge (Car). The said test cartridge (Car) is then transported into the second indication site (L2) where the fluid sample (F) within the global test volume (Vep) of the reaction chamber (Cre) is placed in contact with an active component (chemical and/or biological) known as a receptor (R) [and possibly with another active component known as an indicator (U)]. Then the test cartridge (Car) is transported into a third measurement site (L3). The lateral integral measurement transducer system (T) of the extensive state variable (E) is positioned in the measurement site (L3). In FIG. 19b the first sampling site (L1) and the second indication site 25 (L2) are combined in a common sampling/indication site (L1/L2). In this diagram the test cartridge (Car) remains in the same device for the sampling and indication phases. It is then transported into a separate third measurement site (L3). In FIG. 19c it is the second indication site (L2) and the third measurement site (L3) which are combined in a common indication/measurement site (L2/L3). In FIG. 19d the first sampling site (L1), the second indication site (L2) and the third measurement site (L3) are combined in a common sampling/indication/measurement site (L1/L2/L3). In this diagram, once it is inserted in the common device the test cartridge (Car) is not displaced until the end of the method of analysis.

It is recommended by the invention to coat the cylindrical multi-microtubular reaction chamber (Cre), and more easily the test cartridge (Car) with an identifier (Id) prior to the displacement in order to ensure the traceability thereof. A preferred variant of this identifier is the barcode label (83) shown in FIGS. 5a and 6a.

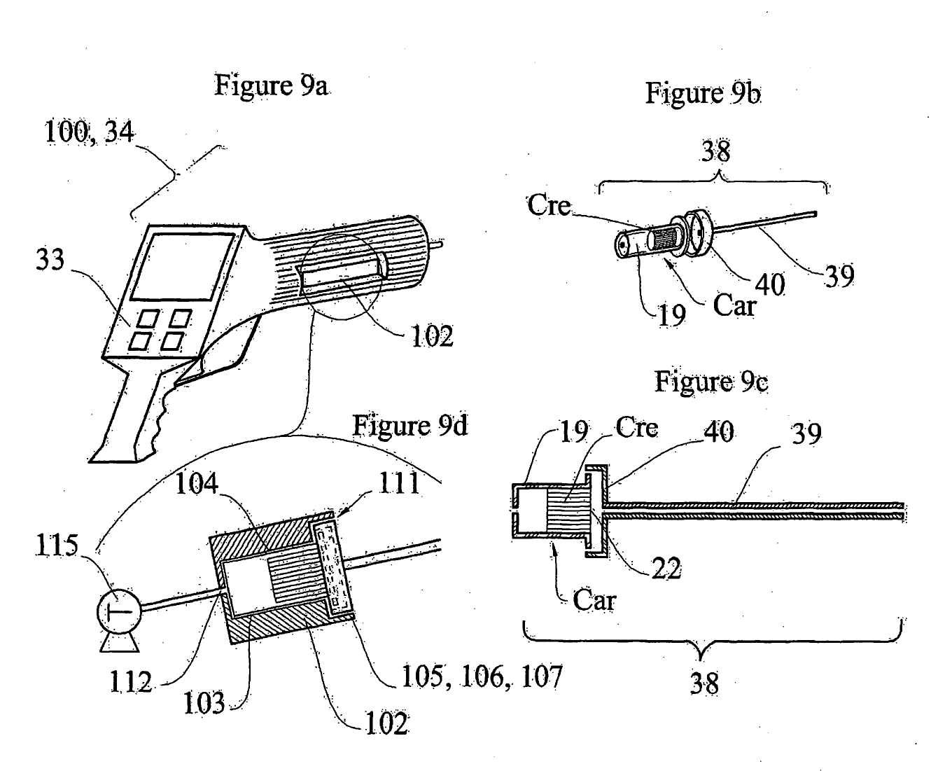

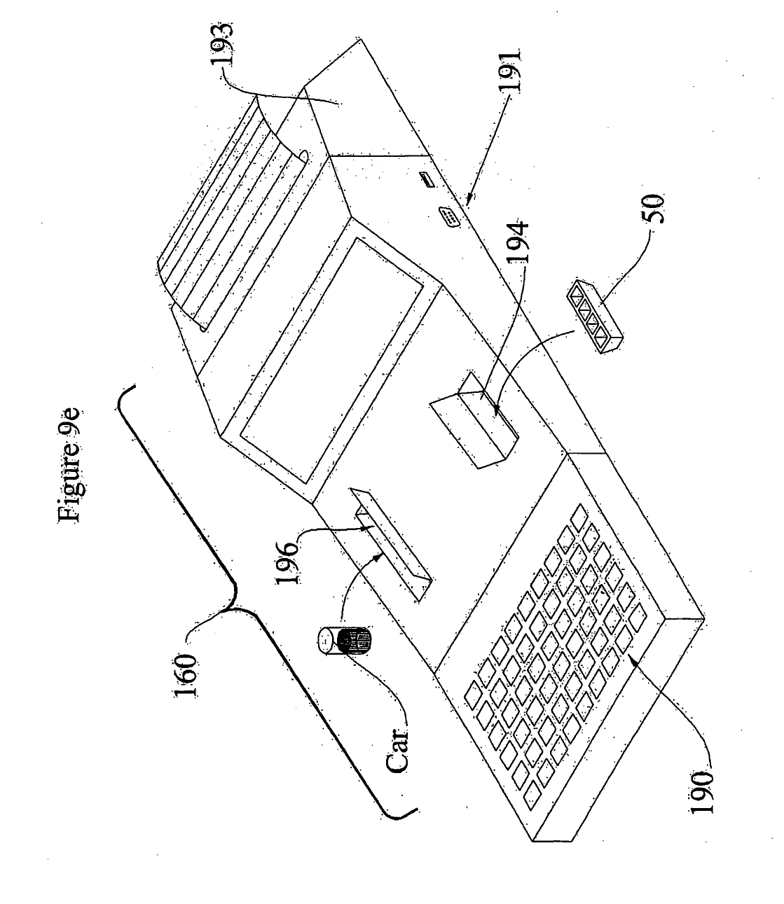

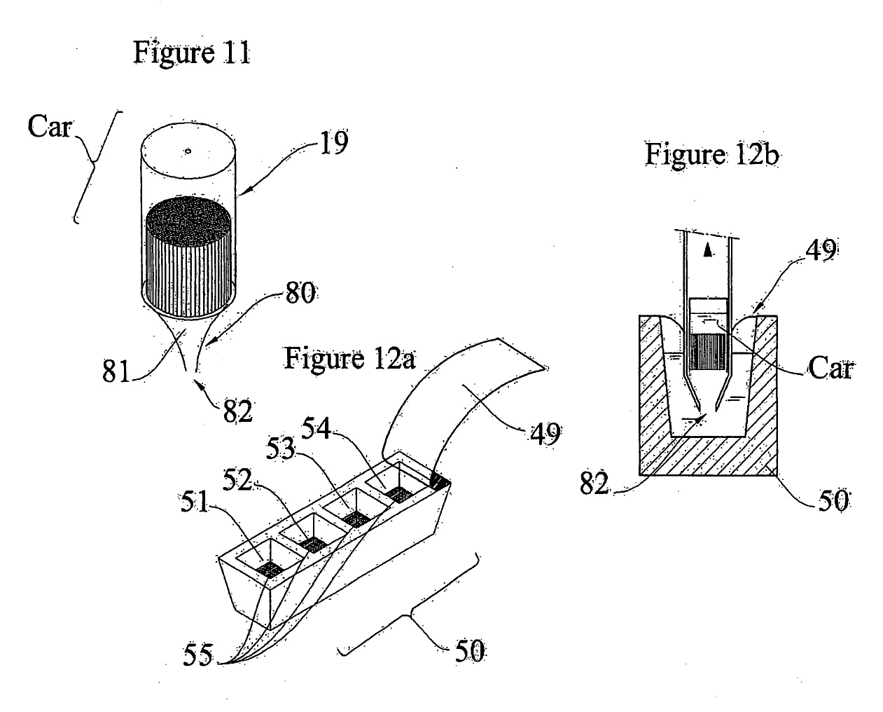

FIGS. 9a to 9e describe schematically a multi-location sensor (in two parts) of the type of operation described in FIG. 19c. In a first sampling site (L1) a mobile sampling device (100) serves for sampling of the fluid sample (F) in a mobile test cartridge (Car). In the example described the mobile sampling device (100) is a sampling gun (34) shown in FIG. 9a. The sampling gun (34) comprises a sampling block (102) having an internal sampling cavity (103) of revolutionary shape (cylindrical or truncated cone). A rebate (107) disposed upstream the sampling block (102) simultaneously constitutes a retaining means (105) and a sealing means (106) of the mobile cartridge (Car). After introduction of the test cartridge (Car) the sampling block comprises two openings: an opening upstream (111) for sampling the fluid sample (F) and an opening downstream (112). Finally, a pump (115) for movement of the fluid sample (F) is connected to one or the other of the upstream sampling opening (111) or downstream opening (112). The sampling gun (34) uses needle cartridges (38) described in FIG. 9c. A sampling needle (39) equipped with a cover (40) is fitted in a sealed and removable manner on the protective casing (19) facing the upstream face (22) of a cartridge (Car). It is situated on the side of the permeable upstream front face (sfam) of the reaction chamber (Cre). The needle cartridge (38) is introduced into the block (102) of the sampling gun (34). In order to remove a portion of fluid sample (F) the trigger of the gun is pressed. The needle cartridge (39) is displaced towards the exterior of the barrel of the gun, so that the needle (39) is visible. The fluid sample (F) is drawn in through the needle (39) and is multi-channelled in parallel through the reaction chamber (Cre) of the needle cartridge (38). The needle is then disengaged from its cartridge (Car) and collected in a receptacle for used needles. In a variant shown in FIG. 11, the protective casing (19) of a test cartridge (Car) can be extended upstream of its upstream end face (22) in a sampling cone (80) equipped with a sampling cavity (81) in its end (82). In another variant a standard test cartridge (Car) can be used.

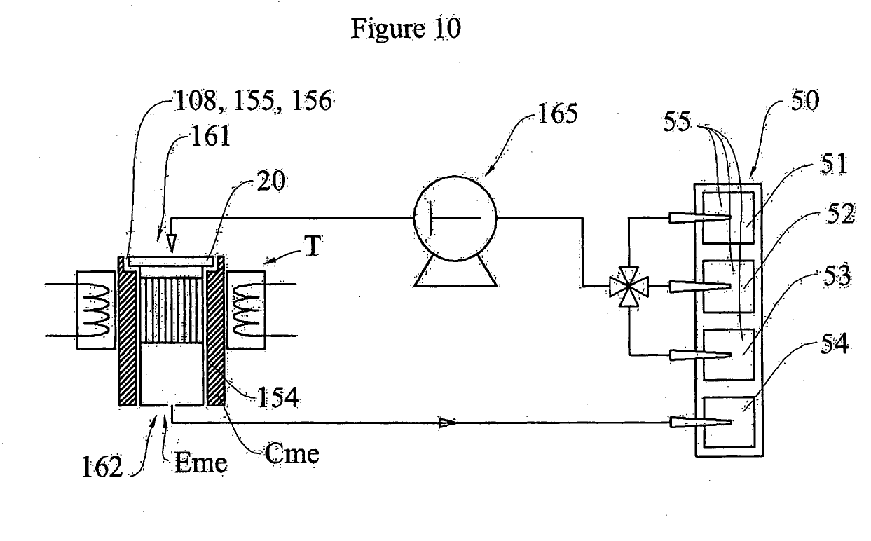

Then the cartridge (Car) is removed from the sampling gun (34). In an independent device for indication and measurement (160) described in FIGS. 9e and 10, the mobile test cartridge (Car), including the reaction chamber (Cre) in the form of a multi-microtubular array is introduced via a first receptacle (196), inside the cylindrical internal measurement cavity (Eme) of the measurement block (Cme). As is apparent in greater detail in FIG. 10, a shoulder (108) of the block (Cme) co-operates with the annular tongue (20). It serves as a means for retaining (155) the mobile cartridge (Car) and as a means for sealing (156) the block (Cme) after introduction of the mobile cartridge (Car) relative to the wall (154) of the internal measurement cavity (Eme). The block (Cme) has an upstream opening (161) for introduction of the fluids (55) and a downstream opening (162). A pump (165) for movement of the fluid samples and/or reagents is connected to upstream sampling opening (161). Then a strip of wells (50) [such as is shown in FIG. 12a] containing the fluids (55) [reagents and washing solutions] necessary for an analysis according to the rules of the art is introduced into a second receptacle (194) situated inside the independent device for indication and measurement (160). Typically this strip made from rigid plastics material comprises four independent wells (51, 52, 53, 54) closed by a cover (49) formed by a sheet of plastics material. The first well (51) contains a washing solution constituted by a buffer at pH 7.0. The second well (52) contains the receptor elements (rⱼ), [in this case a suspension of secondary antibodies (asⱼ), specific to the sought analyte, for example Cryptosporidium]. These antibodies are grafted with super-magnetic micro-granules(spⱼ). The third well (53) contains a washing solution constituted by a buffer at pH 7.0. The fourth well (54) is empty. It serves as a waste bin for the used reagents. This strip (50) is thrown away after the analysis. The washing solutions and the reagents are successively multi-channelled in parallel through the reaction chamber (Cre) of the test cartridge (Car) with the aid of a pump (165). This takes place according to a process program digitally recorded in an EPROM memory previously programmed as a function of the nature of the analyte elements sought. At the end of the execution of this program the analyte elements (aᵢ), in this case Cryptosporidium, immobilised on the test surface (Sep) are marked by the receptor elements (rⱼ).

Then by means of the lateral integral measurement transducer system (T) an integral measurement of the variations of the said extensive state variable (E) is carried out through both the substantially cylindrical external lateral surface (Secm) of the periphery of the measurement block (Cme), the lateral wall (Cpl) of the test cartridge (Car), and the impermeable lateral face (slat) of the reaction chamber (Cre).

The traceability of the test cartridges (Car) between the sampling site(L1), in this case the sampling gun (34), and the indication/measurement site (L2/L3), in this case the independent device for indication and measurement (160), is ensured by an identification label (83) of the test cartridge (Car) with a barcode of the type described in FIG. 5a. The sampling gun (34) is equipped with a keypad (33) which permits the capture of the specific data of the fluid sample (F) removed and with a Wifi-type system of emission to a centralised database. The independent device for indication and measurement (160) is itself connected to this database, receives from it and sends to it the data relating to the analysis referenced by the barcode of the identification label (83) of the test cartridge (Car). The independent device for indication and measurement (160) can be equipped with a printer (193) and a keypad (190) or can be directly connected to a computer by an input/output port (191).

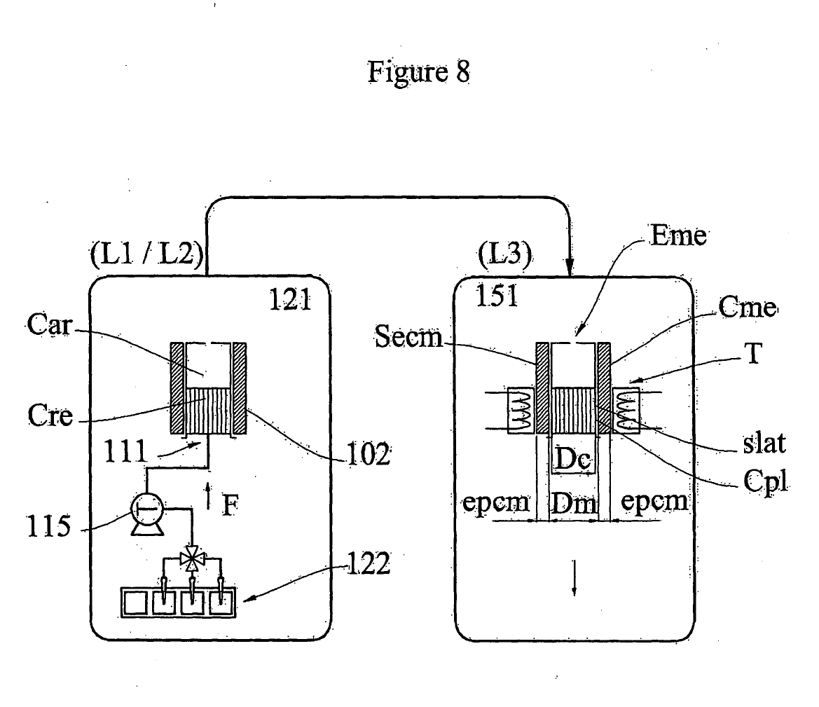

FIG. 8 describes schematically the method of operation according to the invention of a multi-location sensor (in two parts) of the type described in FIG. 19b. The first sampling site (L1) and the second indication site (L2) are merged in a common sampling/indication site (L1/L2). In this variant a mobile device for sampling and indication (121) by mobile test cartridge (Car) is adapted to be used with the sampling gun (34) by the addition of at least one reservoir (122) of chemical and/or biological reagent. The reservoir (122), in this case a strip for reagents and washing solutions adapted to the strip of wells (50) described in FIG. 12a, is connected to the test cartridge (Car) by the upstream sampling opening (111) of the sampling block (102) by means of a pump for movement of the fluids (115). The mobile test cartridge (Car) is then transferred in the measurement site (L3) into an independent measurement device (151) shown schematically in FIG. 8. The cartridge (Car) is introduced into the cylindrical internal measurement cavity (Eme) of the measurement block (Cme) of thickness (epcm). The measurement block (Cme) has a diameter (Dm) substantially equal to but strictly greater than the cartridge diameter (Dc). As described previously for the indication and measurement device (160), by means of the lateral integral measurement transducer system (T) an integral measurement of the variations of the said extensive state variable (E) is carried out, simultaneously through the substantially cylindrical lateral external surface (Secm) of the periphery of the measurement block (Cme), the lateral wall (Cpl) of the test cartridge (Car), and the impermeable lateral face (slat) of the reaction chamber (Cre).

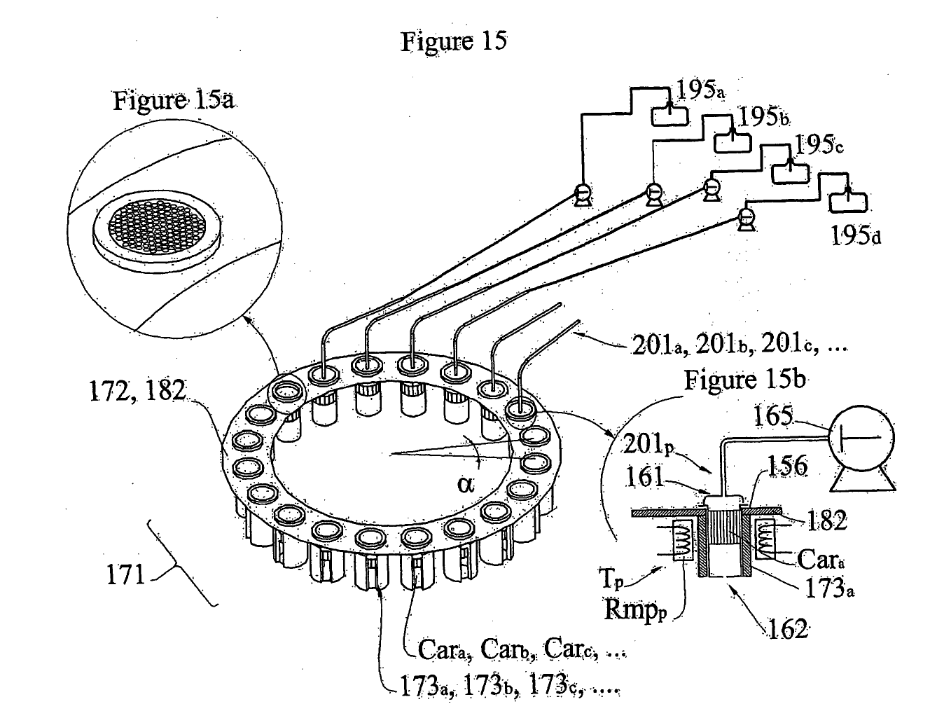

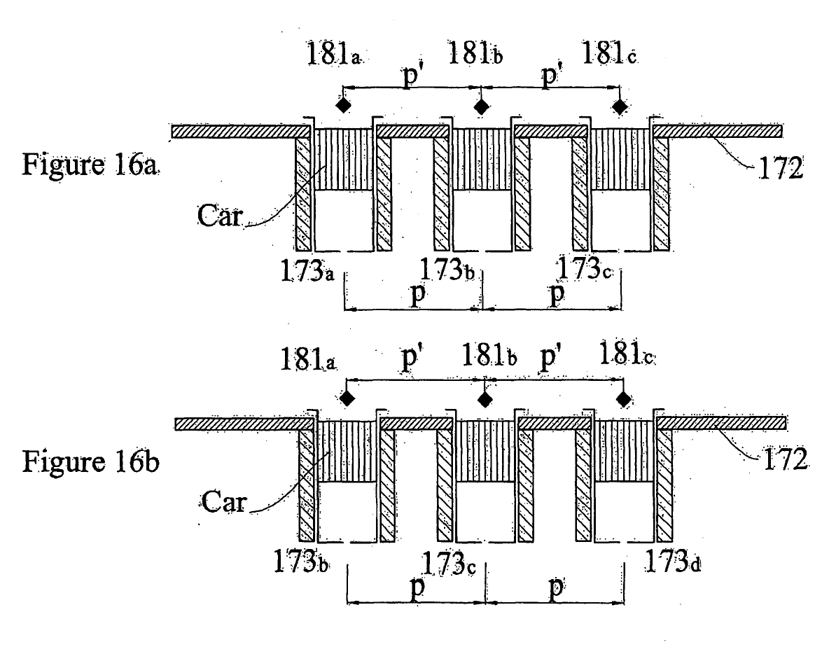

The method of operation described in FIG. 19a of a multi-location sensor (in three parts) according to the invention is carried out broadly on the basis of the two examples described above. In a first sampling site (L1) a mobile sampling device (100), preferably the sampling gun (34), is used. In a third measurement site (L3) the independent measurement device (151) described in FIG. 8 is used. In the second indication site (L2) an independent device for indication after sampling (131) is used. After sampling, the mobile test cartridge (Car) with a revolutionary shape (cylinder or truncated cone) is introduced into the internal indication cavity of a indication block of revolutionary shape (cylinder or truncated cone) which is complementary to that of the test cartridge. The retention of the cartridge mobile in the indication block, the sealing of the indication block after introduction of the mobile cartridge (Car) inside the internal indication cavity, and the movement of the sample fluids and reagents are ensured as for the independent indication and measurement device (160) described in FIG. 10. A variant of the preferred embodiment of the method according to the invention, in the form of a multi-location sensor adapted for the automated processing of a large number of samples is shown in FIGS. 15, 15a and 15b, 16a and 16b. It also comprises two parts. A sampling device, which may be the sampling gun (34) described previously, serves for sampling of the fluid sample (F) in a test cartridge (Car). A sequential robot device (171) for analysis after sampling by mobile test cartridge (Car) is based on a carousel (182). It comprises a rigid cartridge support (172), comprising in this example precisely 20 blocks (173ₐ, 173_{b}, 173_{c}, 173_{d}, . . . ) positioned on the periphery of the carousel (182) and separated by an equal angle at the top (α), in this case equal to 18°, which constitutes the constant pitch (p) for spacing of the blocks. Each block has a sealing means (156) which is active after introduction of the mobile cartridge (Car) into the interior. It has two openings, an upstream opening (161) for feeding, and a downstream opening (162). A pump (165) for movement of the fluid samples and/or reagents is connected to the upstream opening (161). A means for periodic displacement of the carousel (182), in this case an electric motor, displaces the plurality of blocks (173ₐ, 173_{b}, 173_{c}, 173_{d}, . . . ) by a spacing (p′) equal to the said constant pitch (p) facing an identical plurality of stopping points (181ₐ, 181_{b}, 181_{c}, . . . ) by periodic rotation of the carousel (182) by an angle (α). Twenty mobile test cartridges (Carₐ, Car_{b}, Car_{c}, Car_{d}, . . . ) each including a reaction chamber (Creₐ, Cre_{b}, Cre_{c}, Cre_{d}, . . . ) in the form of a monolithic multi-microtubular array, are inserted into the interior of the plurality of blocks (173ₐ, 173_{b}, 173_{c}, 173_{d}, . . . ). The carousel (182) is equipped with a device for injection of liquid (201ₐ, 201_{b}, 201_{c}, . . . ) situated facing the stopping point(s) (181ₐ, 181_{b}, 181_{c}, . . . ). This device is equipped with a plurality of independent reservoirs (195ₐ, 195_{b}, 195_{c}, . . . ) for the washing solutions and suspensions of reagents which can be used for several types of analytes, for example Salmonella, Legionella, Cryptosporidium. The protocols and the choice of the reagents to be multi-channelled (which may be pre-programmed in the microprocessor of the apparatus) are carried out as a function of the indications provided by means of the barcode identification label (83) of the test cartridge (Car). The device comprises at least one physical measurement receiver (Rmp₁, Rmp₂, Rmp₃, . . . , Rmpₚ, . . . ) [such as in particular a magnetic field receiver (13)] which is positioned at a stopping point (181ₐ, 181_{b}, 181_{c}, . . . ), periodically movable perpendicular to the movement of the cartridge support (172), and comes periodically to mount the test cartridge situated facing it, at the stopping point (181ₐ, 181_{b}, 181_{c}, . . . ), closely surrounding the external surface thereof. In this example the physical measurement receiver (Rmp₁, Rmp₂, Rmp₃, . . . , Rmpₚ, . . . ) is the active part of a lateral integral measurement transducer (T₁, T₂, T₃, . . . , Tₚ, . . . ).

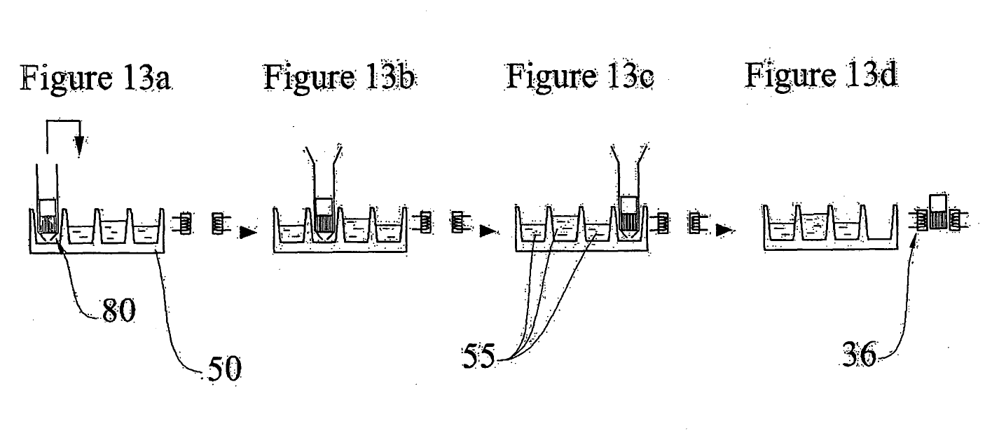

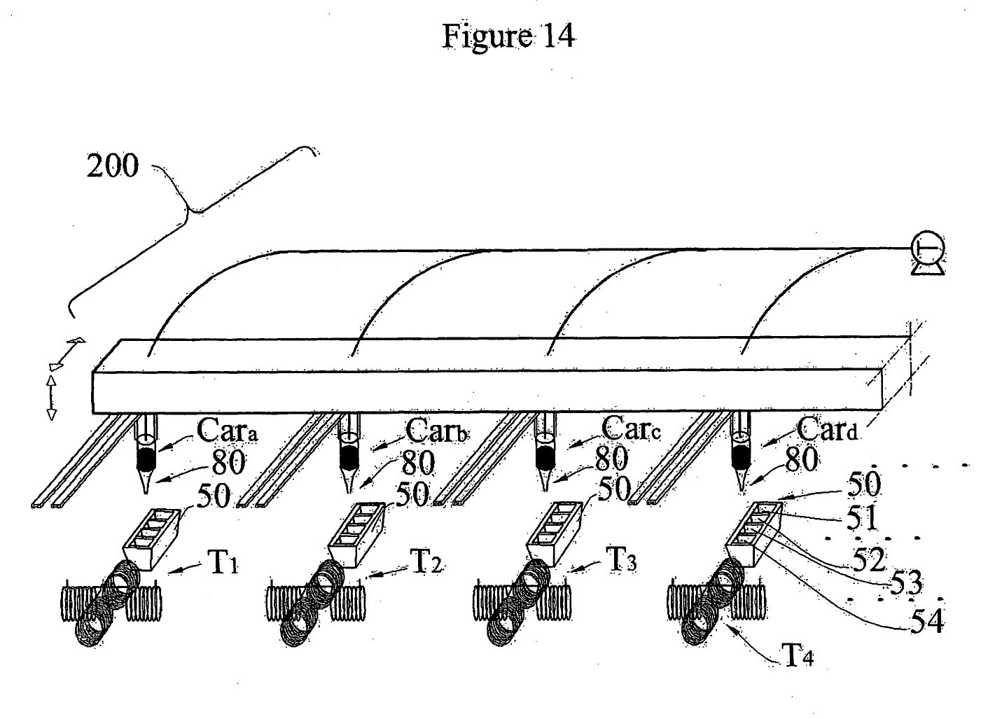

Another mode of operation for the multi-location evaluation of the concentration of analyte elements (aᵢ) of an analyte (A) is described in FIGS. 13a to 13d. The mobile test cartridge (Car) is successively immersed in the interior of a succession of wells (51, 52, 53, 54) containing different fluids (55) such as the fluid sample (F) and/or reagents and washing solutions. After each introduction into a well (51, 52, 53, 54), a fraction of the fluid (55) from the well (51, 52, 53, 54) is drawn in and multi-channelled through the reaction chamber (Cre) in the form of a multi-microtubular array of the cartridge (Car). Moreover, after each intake of the fluid (55) from a well (51, 52, 53, 54), through the reaction chamber (Cre) this fluid (55) from the different microtubular channels (cₖ) is forced towards the same well (51, 52, 53, 54). An independent linear robot device for indication/measurement (200) based on this mode of operation is described in FIG. 14. The test cartridges (Carₐ, Car_{b}, Car_{c}, . . . ) are introduced into the interior of this device (200) which can accommodate several of them for simultaneous processing, typically 16. Likewise multi-well strips (50) of the type described previously are introduced into the interior of the device in an identical quantity to the test cartridges (Carₐ, Car_{b}, Car_{c}, . . . ), typically 16. A motor ensures a lateral movement of the support of the test cartridges (Carₐ, Car_{b}, Car_{c}, . . . ) in order to displace them from one well to the other. It also ensures the vertical movement of the test cartridges (Carₐ, Car_{b}, Car_{c}, . . . ) in order to draw in and force through the fluids (55). Several fluid samples can be analysed simultaneously but with the same analyte (A) being sought in all the test cartridges (Carₐ, Car_{b}, Car_{c}, . . . ). The test cartridges and the strips must therefore all be of the same type, for example for seeking Cryptosporidium. In the case described, at the end of the indication process the test cartridges (Carₐ, Car_{b}, Car_{c}, . . . ) are introduced by means of the motor into the measurement blocks of lateral integral measurement transducers (T₁, T₂, T₃, . . . , Tₚ, . . . ) as described above.

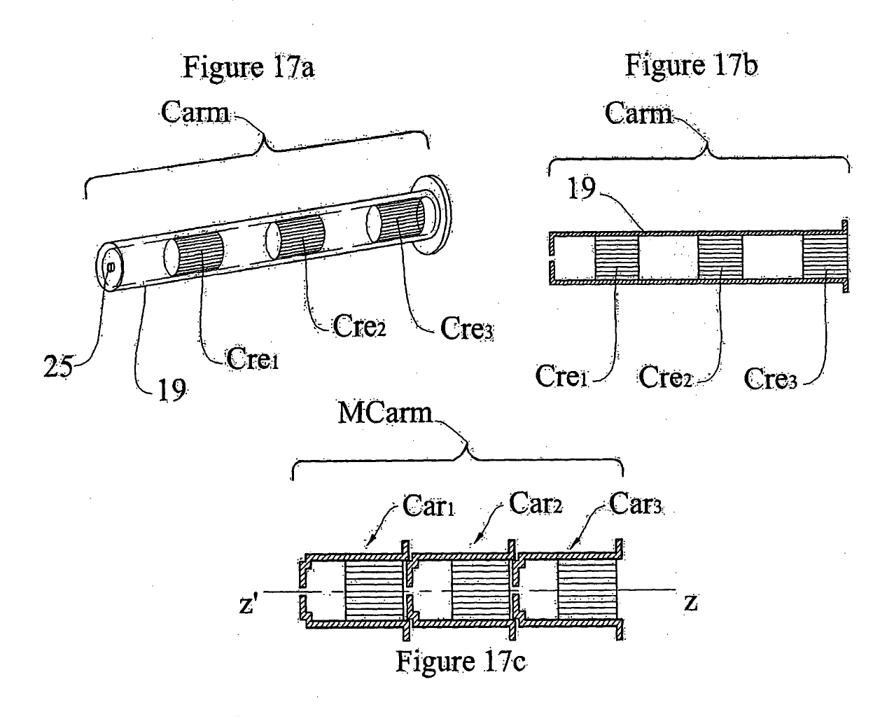

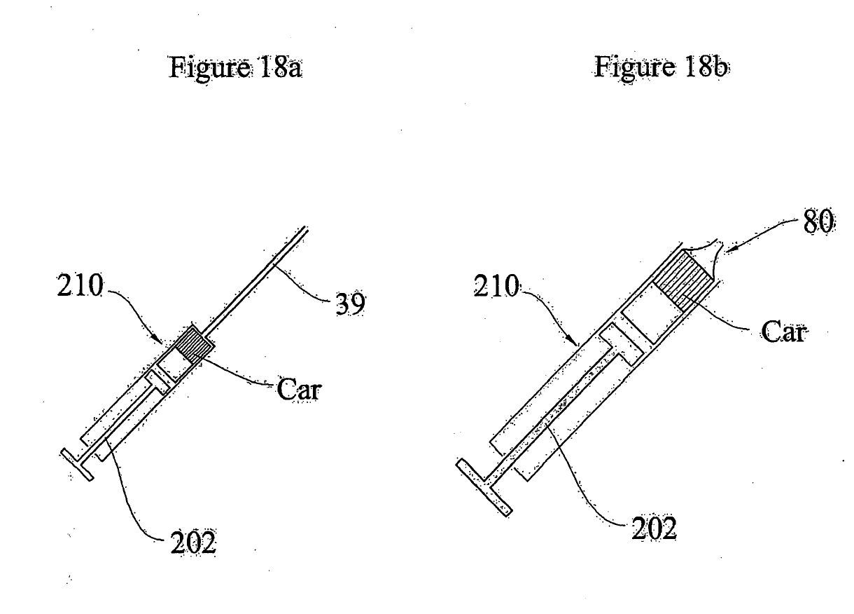

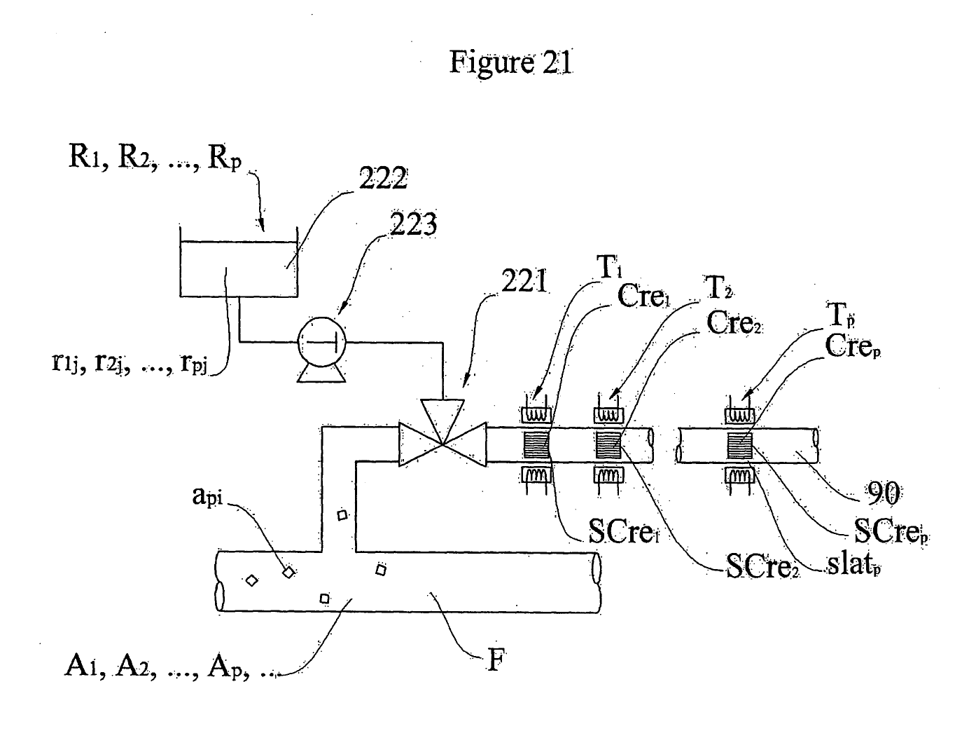

Another variant of the preferred embodiment for multi-location evaluation of the concentration of analyte elements (aᵢ) of an analyte (A) relates to the mobile device for sampling of the fraction of fluid sample (F). The sampling gun (34) is replaced by a sampling syringe (210). This single-use syringe is equipped with a test cartridge (Car) through which the fluid sample is multi-channelled by suction when its piston (202) is actuated. For taking the fluid sample (F) the syringe can be equipped either with a needle (39) in FIG. 18a or with a sampling cone (80) in FIG. 18b. The test cartridge is then withdrawn from the syringe in order to be processed according to the preferred embodiment or the variants thereof presented above. Another preferred embodiment in the form of a monobloc multi-analyte bio-sensor is presented in FIG. 21. In the example described below it is used to seek simultaneously bacteria Cryptosporidium, the analyte (A₁), Escherichia coli, the analyte (A₂), and Legionella, the analyte (A₃), in a fluid sample (F) [in this case the water present in the distribution channels]. It is constituted by a multi-stage reactor tube (90). Three reaction chambers (Cre₁, Cre₂, Cre₃) each formed by an array (18) of a plurality of cylindrical micro-tubular channels (cₚ₁, cₚ₂, . . . , cₚₖ, . . . , cₚₙ) are disposed inside this multi-stage reactor tube (90) coaxially in series and and so as to be sealed laterally. Strictly outside of the multi-stage reactor tube (90) there are disposed three lateral integral measurement transducer systems (T₁, T₂, T₃) of which the windings fit over the corresponding reaction chamber (Cre₁, Cre₂, Cre₃), facing the corresponding impermeable lateral face (slat₁, slat₂, slat₃). In this precise example the test surfaces of the reaction chambers have been previously coated, the reaction chamber (Cre₁) with an antibody specific to the bacterium Cryptosporidium, the reaction chamber (Cre₂) with an antibody specific to the bacterium Escherichia coli, and the reaction chamber (Cre₃) with an antibody specific to the bacterium Legionella. A fraction of the fluid sample (F) is multi-channelled inside the multi-stage reactor tube. The analyte elements (aₚᵢ), in this case the bacteria Cryptosporidium, Escherichia coli, or Legionella, if they are present, are fixed specifically on the test surface: Cryptosporidium in the reaction chamber (Cre₁), Escherichia coli in the reaction chamber (Cre₂), Legionella in the reaction chamber (Cre₃). Then the multi-stage reactor tube (90) is supplied by means of a pump (223) via a three-way valve (221) with a mixture (R₁, R₂, R₃) of antibodies grafted with specific super-magnetic micro-granules (spⱼ), (R₁) of Cryptosporidium, (R₂) of Escherichia coli, (R₃) of Legionella contained in a multi-reagent reservoir (222). The grafted antibodies are fixed specifically, (R₁) in the reaction chamber (Cre₁), (R₂) in the reaction chamber (Cre₂), and (R₃) in the reaction chamber (Cre₃). Finally the multi-stage reactor tube (90) is washed by passer water (F) through via the three-way valve (221). The disturbance measured in each reaction chamber (Cre₁, Cre₂, Cre₃) by each lateral integral measurement transducer system (T₁, T₂, T₃) is linked to the concentration of bacteria Cryptosporidium pour (T₁), of bacteria Escherichia coli for (T₂), of bacteria Legionella for (T₃), present in the fluid sample (F). A variant of the test cartridge (Car) can be used. This is a multi-chamber test cartridge (Carm) which is illustrated in FIGS. 17a and 17b in perspective and in section. It comprises at least two reaction chambers (Cre₁, Cre₂, . . . ) in the form of a multi-microtubular array, of identical cross-section, positioned in the axis (zz′). These reaction chambers are covered by a single protective casing (19). The multi-chamber test cartridges (Carm) are used for the simultaneous detection of at least two different analytes (A₁, A₂, . . . ) present in the fluid sample (F). Each reaction chamber (Cre₁, Cre₂, . . . ) is specific to one analyte. The mode of use of the multi-chamber test cartridges (Carm) is based broadly upon that of the multi-stage reactor tube (90). Another variant is the multi-chamber multi-test cartridge (MCarm) formed by a plurality of test cartridges (Car₁, Car₂, Car₃, . . . ) in accordance with the general description which are disposed end to end in series along one and the same axis (zz′) and fitted into one another two by two according to FIG. 17c. The indication and/or measurement devices must be adapted to this type of multi-chamber cartridge whilst adhering to the spirit of the invention as described above.

Although the invention is described and illustrated in detail here for certain examples of application in order to aid understanding, it is clear to a person skilled in the art that certain modifications can be made to these examples without departing from either the spirit or the scope of the claims of the invention.

### OBJECTS AND ADVANTAGES OF THE INVENTION

The principal object of the combination of monolithic reaction chambers in the form of a multi-microtubular array with a lateral integral transduction is to concentrate a large number of recognition events into one very compact test volume, and thus to be able to obtain a homogeneous and sufficiently strong signal from outside the test volume.

In greater detail the advantages are as follows:
1) Increasing the ratio “per unit area” [between the test surface of the reaction chamber and its mean test section] and thereby even to increase the density per unit volume of events of recognition of analyte elements by receptor elements within the test volume;
2) Increasing the “sensitivity” ratio [between the test surface of the reaction chamber and its test volume] and thereby even the efficiency of the transducer and the sensitivity of the sensor;
3) Lowering the sensitivity threshold of a sensor, thus avoiding the prior phase of enrichment of the sample or enzymatic amplification of the recognition events, and thus obtaining truly rapid sensors;
4) Focussing the analyte elements or the active components close to the test surface, and thus accelerating their bonding kinetics, traditionally a limiting factor in immunological recognition or in hybridisation of nucleic acids. In fact the receptor elements and analyte elements have a very strong affinity with a thermodynamic constant K of the order of 10³⁰. However, bonding thereof, of the “key/lock” type, necessitates perfect alignment a small distance apart of the specific recognition sites. In order to reduce, on average over the entire population of elements, the energy of activation of the bond, and to render it possible in the normal conditions of temperature and of solvent, it is necessary to increase the probability of this alignment a small distance apart. The micro-tubular geometry precisely minimises the average test distance of the elementary portions of the flow at the test surface, and on the other hand makes it possible to reduce the average test speed of the elementary portions of the flow in order to increase their residence time close to the test surface;
5) Decreasing the dispersion of behaviour of the portions of fluids (sample, reagents) in all the channels by subjecting them to identical or “quasi-identical” conditions by means of the regularity of the structure of the reaction chamber, in order to decrease the variation of recognition kinetics, and thus to increase the signal to noise ratio of the sensor;
6) Reducing the non-specific coupling of the analyte elements or of the receptor elements, and thus reducing the noise of the sensor. The non-specific coupling of receptor elements takes place either with bacteria close to the ones sought with which the receptor elements may have an affinity which is limited but not zero, or with the solid support itself. This non-specific coupling is more particularly significant in irregular structures based on fibres or agglomerated beads. These structures in effect provide the zones where the slowing down of the flow and the steric dimensions on the one hand more easily enable the non-specific fixing and on the other hand render the washing operations less efficient;
7) Decreasing the size of the test volume and thereby even the dimensions of the reaction chamber of sensor, with equal sensitivity;
8) Decreasing the quantity of fluid sample and of reagents consumed by a sensor, with equal sensitivity, and thereby even facilitating the implementation and reducing the costs of consumables;
9) Decreasing the loss of charge through the test volume and thus limiting the pressure necessary to ensure the displacements of fluids;
10) Separating geometrically the “recognition/indication” zones and the transduction zones of a sensor in order to rationalise the industrial implementation;
11) Producing the reaction chamber of a sensor in the form of a cartridge which can be produced industrially on a large scale, is less cumbersome and is disposable;
12) Simplifying the handling of the fluid samples and reagents and avoiding the handling and the use of the active components by the user;
13) Rationalising the use of a sensor by limiting the number of handling operations;
14) Lowering the cost, the duration and the competent mobilisation of a measurement by sensor;
15) Enabling anyone, even a non-specialist, to use a sensor in order to obtain rapidly and without special training the result of analysis of a sample;
16) Enabling effectively the use within a sensor of clean and ecological indicators (non-radioactive, . . . ) such as super-paramagnetic micro-granules, whilst ensuring a high sensitivity of measurement.

### POTENTIAL INDUSTRIAL APPLICATIONS OF THE INVENTION

The methods and biosensor devices according to this invention are useful for detecting analytes for numerous industries including, but not exclusively, health, agricultural produce, chemicals, the environment. The types of samples may include various fluids such as blood, plasma, urine, saliva, milk, wine, beer, chemical products, liquid effluents, water from watercourses or withdrawn from public or private distribution circuits. In certain instances the sample may be prepared before analysis. If it is initially complex, solid, very viscous or gaseous, it can first of all be extracted, dissolved, diluted, in order to give it the physical characteristics compatible with multi-channelling it in the reaction chamber, and the chemical characteristics compatible with the stability of the test surface and the recognition complexes (for example a pH of between 5 and 9). A large variety of analytes can be detected using the methods and devices according to the invention. These are all the analytes capable of being recognised and of forming a pair with a specific receptor. The analyte may be an antigen, an antibody or a hapten for the smaller molecules such as certain hormones. It may equally be a nucleic acid (DNA or RNA) or an oligonucleotide capable of hybridising with the complementary nucleotide. It may equally be a specific enzyme of certain substrates. Some examples: antibiotics; food additives; micro-organisms such as yeasts, unicellular algae, bacteria, viruses, prions, rickettsiae; toxins, dyes, pathogen markers present in the biological fluids, antibodies, active ingredients of medicaments, cytokines, surface proteins of cellular membranes, etc.

1) Method for the production of a sensor for evaluation of the concentration of analyte elements (aᵢ) of an analyte (A) [that is to say soluble chemical entities or live or dead micro-organisms, or parts of micro-organisms] which are present in a fluid sample (F) included initially in a sample volume (Vec), this method being of the type according to which the following steps are carried out:
a) a fraction of the fluid sample (F) is channelled in the interior of a test volume (Vep),
i) circumscribed by an enclosing reaction surface (Sev) [topologically, the smallest continuous surface surrounding the said test volume (Vep)],
ii) provided internally within a reaction chamber (Cre),
iii) the said enclosing reaction surface (Sev) being constituted by:
a permeable upstream front face (sfam),
permeable downstream front face (sfav) situated opposite the permeable upstream face (sfam),
and a substantially cylindrical impermeable lateral face (slat) connected by its two ends to the peripheries of the said two upstream (sfam) and downstream (sfav) faces;
b) the fluid sample (F) is placed in contact, within the test volume (Vep), with an active component (chemical and/or biological) known as a receptor (R):
i) of which the receptor elements (rⱼ) have an affinity with the analyte elements (aᵢ) in order to detect them,
ii) and moreover having the property [alone or in combination with another active component known as a indicator (U) likewise introduced into the interior of the test volume (Vep)] of modifying by an elementary signal (dE), a measurable extensive state variable (physical and/or chemical) (E), at each occurrence [or according to a certain law of probability], during an event of recognition of an analyte element (aᵢ) by a receptor element (rⱼ),
c) by means of a measurement transducer system (T) the variations in the said extensive state variable (E) are measured in such a way as to quantify the presence of the analyte elements (aᵢ) in the fluid sample (F) in the form of an exploitable analytical signal (Se);
This method of evaluation of concentration being characterised in that in combination:
d) on the one hand, the fraction of the fluid sample (F) is multi-channelled in parallel through a reaction chamber (Cre) in the form of a monolithic multi-microtubular array, that is to say constituted by the joining of a plurality of cylindrical micro-tubular channels disposed substantially parallel and multi-tangent (c₁, c₂, . . . , cₖ, . . . , cₙ),
i) cylindrical [that is to say each delimiting an elementary internal surface (sepₖ) generated topologically by the displacement along an elementary central line of a continuous virtual skeleton (lₖ) of a curve of continuous and closed shape (fₖ) placed substantially perpendicular],
ii) microtubular [that is to say of which the elementary internal section (sₖ) perpendicular to their elementary central line (lₖ) has at least one selective transverse dimension (dx) several orders of magnitude smaller than its length (l) (typically of the order of 1000 times smaller)],
iii) with lengths (l) [that is to say the length of elementary central lines (lₖ)] which are substantially equal,
iv) disposed substantially parallel [that is to say of which the elementary central lines (lₖ) are disposed substantially parallel],
v) and multi-tangent [that is to say of which each micro-tube (cₖ) is in longitudinal contact over substantially all its length with at least one other adjacent micro-tube (cₖ′)],
vi) thus delimiting a dense plurality of adjacent separate convex elementary volumes (vec₁, vec₂, . . . , vecₙ) which are open at their two ends and of which the joining constitutes the non-convex global test volume (Vep), this latter being circumscribed by the enclosing reaction surface (Sev) of which the permeable upstream (sfam) and downstream (sfav) front faces are situated at right angles to the inlet (seₖ) and outlet (ssₖ) sections of the microtubular channels (c₁, c₂, . . . , cₖ, . . . , cₙ);
e) on the other hand, the lateral integral measurement transducer system (T) for the extensive state variable (E) is positioned laterally to the reaction chamber (Cre), that is to say
i) entirely outside the enclosing surface (Sev) of the reaction chamber (Cre),
ii) and strictly facing the impermeable lateral face (slat),
f) finally, by means of the lateral integral measurement transducer system (T) an integral measurement ΔE=Σ_{k=1 . . . n} Σᵢⱼ (dE)ᵢⱼₖ is carried out, that is to say
i) a summation of the variations (dE)ᵢⱼₖ in the said extensive state variable (E),
ii) simultaneously for all the elementary volumes (vecₖ) at once,
iii) and for all the elementary signals (dE)ᵢⱼₖ in each elementary tube (cₖ) at once,
iv) through the impermeable lateral face (slat), in such a way as to quantify globally the presence of the analyte elements (aᵢ) in the fluid sample (F) in all the microtubular channels (c₁, c₂, . . . , cₖ, . . . , cₙ) at once.

2) Method according to claim 1 for the production of a sensor for evaluation of the concentration of analyte elements (aᵢ) of a biological analyte (A), constituted by live or dead micro-organisms (aᵢ) [such as microscopic fungi, bacteria, or parts of micro-organisms] of typical diameter (dt) [typically situated between 0.01 microns and 10 microns], this method being characterised in that furthermore:
a) the fraction of the fluid sample (F) charged with biological analyte elements (aᵢ) is multi-channelled in parallel through a reaction chamber (Cre) in the form of a monolithic multi-microtubular array constituted by the joining of a plurality of microtubular channels (c₁, c₂, . . . , cₖ, . . . , cₙ), of which the said selective transverse dimension (dx) is chosen in correlation with the typical diameter (dt) of the biological analyte elements (aᵢ), [typically the elementary internal section (sₖ) of the microtubular channels (cₖ) is chosen in such a way that their said transverse dimensions (dx) are substantially equal to approximately 10 times the typical diameter (dt) of the biological analyte elements (aᵢ), that is to say in particular of the order of magnitude of 10 microns if the biological analyte elements (aᵢ) are bacteria].

3) Method according to claim 1, for the production of a sensor for evaluation of the concentration of analyte elements (aᵢ) of an analyte (A), this method being characterised in that furthermore in combination:
a) on the one hand, the fraction of the fluid sample (F) charged with analyte elements (aᵢ) is multi-channelled in parallel through a reaction chamber (Cre) in the form of a bi-periodic monolithic multi-microtubular array (Cre) constituted by the joining of a plurality of n (n≅approximately 300 000) microtubular channels (cₖ),
i) of quasi-revolutionary cross-section [that is to say a cross-section with a curve of continuous shape (fₖ) such as a circle, ellipse, polygon, oval, . . . , of which any pair of two perpendicular transverse dimensions (dx, dy) are of the same order of magnitude (d)]
ii) of internal transverse dimensions (d=dx=dy≅of the order of 10 microns) which are substantially identical,
iii) disposed parallel, adjacent and joined in a common axial direction (zz′) of orientation of their elementary central lines (lₖ),
iv) and organised according to a bidimensional periodic network (Rxy) perpendicular to the said common axial direction (zz′) of orientation,
b) on the other hand, the said lateral integral measurement transducer system (T) for the extensive state variable (E) is positioned
i) substantially surrounding the exterior of the impermeable lateral face (slat),
ii) at a radial distance (Re) of the order of magnitude of Re≅(2,1*√(n/π)*d) from the axis constituted by the said common direction (zz′) of orientation of the reaction chamber (Cre) (that is to say Re≅7 mm).

4) Method according to claim 1, for the production of a sensor for evaluation of the concentration of analyte elements (aᵢ) of an analyte (A), this method being characterised in that furthermore in combination:
a) on the one hand, the fraction of the fluid sample (F) charged with analyte elements (aᵢ) is multi-channelled in parallel through a reaction chamber (Cre) in the form of a monoperiodic lamellar monolithic multi-microtubular array (Crel), constituted by the joining
i) of a plurality of n (n≅approximately 1 000) microtubular channels (cₖ),
ii) with lamellar cross-sections (selₖ) [that is to say a cross-section with a curve of substantially rectangular shape (fₖ), of which two perpendicular transverse dimensions (dx, dy) are different by at least one order of magnitude (dx<<dy)], typically:
a selective transverse dimension (dx) is of the order of 10 microns,
a lateral transverse dimension (dy) is of the order of 10 millimetres,
iii) disposed parallel, adjacent and joined in a common planar direction (yOz) of orientation of their elementary central lines (lₖ),
iv) according to a monodimensional periodic network (Rx) perpendicular to the said common planar direction (yOz) of orientation,
b) on the other hand, the said lateral integral measurement transducer system (T) for the extensive state variable (E) is positioned substantially surrounding the exterior of the impermeable lateral face (slat).

5) Method according to claim 1, for the production of a sensor for evaluation of the concentration of analyte elements (aᵢ) of an analyte (A), this method being characterised in that furthermore in combination:
a) on the one hand, the fraction of the fluid sample (F) charged with analyte elements (aᵢ) is multi-channelled in parallel through a reaction chamber (Cre) constituted by the joining
i) of a plurality of n substantially identical microtubular channels (c₁, c₂, . . . , cₖ, . . . , cₙ),
ii) parallel and adjacent, which have been disposed transversely and joined in the form of an array constituting a substantially cylindrical bidimensional periodic network (Rxy), such that the impermeable lateral face (slat) of the reaction chamber (Cre) has substantially the shape of a cylinder (Cyre) with a circular cross-section of diameter (De),
b) on the other hand, the said lateral integral measurement transducer system (T) for the extensive state variable (E) is positioned surrounding the exterior of the cylindrical impermeable lateral face (slat) in a substantially annular fashion in such a way as to optimise the ratio of efficiency of measurement (ref=n/Re) between:
i) the number (n) of microtubular channels,
ii) and the distance (Re) between the said lateral integral measurement transducer system (T) for the extensive state variable (E) and the axis (zz′) of the reaction chamber (Cre).

6) Method according to claim 5, for the production of a sensor for multi-location evaluation of the concentration of analyte elements (aᵢ) of an analyte (A), this method being characterised in that furthermore:
a) in a first sampling site (L1),
i) the fraction of the fluid sample (F) charged with analyte elements (aᵢ) is multi-channelled in parallel through a reaction chamber (Cre) in the form of a monolithic cylindrical multi-microtubular array, in the form of a mobile test cartridge (Car), constituted by
the joining of a plurality of n substantially identical microtubular channels (cₖ),
of which the impermeable lateral face (slat) has substantially the shape of a cylinder (Cyre) of cartridge diameter (Dc),
b) in a second indication site (L2), (possibly merged with the first or the third)
i) inside the global test volume (Vep) of the reaction chamber (Cre) the fluid sample (F) is placed in contact with
an active component (chemical and/or biological) known as a receptor (R) of which the receptor elements (rⱼ) have an affinity with the analyte elements (aᵢ) in order to detect them,
and possibly with another active component known as a indicator (U), possibly merged with the receptor (R),
in such a way as to modify by an elementary signal (dE) a measurable extensive state variable (physical and/or chemical) (E), at each occurrence or according to a certain law of probability, of the recognition of an analyte element (aᵢ) by a receptor element (rⱼ) within the test volume (Vep),
c) in a third measurement site (L3), separate from the first,
i) the said lateral integral measurement transducer system (T) for the extensive state variable (E) is disposed around the external lateral surface (Secm) of the periphery of a measurement block (Cme) which is substantially cylindrical and of small thickness (epcm), providing a cylindrical internal measurement cavity (Eme) with a measurement block diameter (Dm) greater than but substantially equal to the said cartridge diameter (Dc),
ii) the mobile test cartridge (Car), including the reaction chamber (Cre) in the form of a multi-microtubular array, is introduced into the interior of the cylindrical internal measurement cavity (Eme) of the measurement block (Cme),
iii) finally, by means of the lateral integral measurement transducer system (T) an integral measurement of the variations of the said extensive state variable (E) is carried out simultaneously through
the external lateral surface (Secm) of the measurement block (Cme),
the lateral wall (Cpl) of the test cartridge (Car),
and the impermeable lateral face (slat) of the reaction chamber (Cre).

7) Method according to claim 6 for the production of a sensor for multi-location evaluation of the concentration of analyte elements (aᵢ) of an analyte (A), this method being characterised in that furthermore in the first sampling site (L1) and/or second indication site (L2),
a) the mobile test cartridge (Car) is immersed successively into the interior of a succession of wells (51, 52, 53, 54) containing different fluids (55) such as the fluid sample (F) and/or reagents which at least one is a receptor (R) of the analyte (A),
b) and after each introduction into a well (51, 52, 53, 54), a fraction of the fluid (55) within the well (51, 52, 53, 54) is drawn in and multi-channelled through the reaction chamber (Cre) in the form of a multi-microtubular array.

8) Method according to claim 7 for the production of a sensor for multi-location evaluation of the concentration of analyte elements (aᵢ) of an analyte (A), this method being characterised in that furthermore in the first sampling site (L1) and/or second indication site (L2), and after each intake of a fluid (55) inside a well (51, 52, 53, 54), through the reaction chamber (Cre) this fluid (55) from the different micro-tubular channels (cₖ) is forced towards the same well (51, 52, 53, 54).

9) Method according to claim 6 for the realisation of a sensor for multi-location evaluation of the concentration of analyte elements (aᵢ) of an analyte (A), this method being characterised in that furthermore:
a) on the one hand, the impermeable lateral face (slat) of the reaction chamber (Cre) in the form of a monolithic multi-microtubular array is given
i) the overall shape of a quasi-cylinder (Cyre) of cartridge diameter (Dc),
ii) of slightly truncated conical shape, with an angle at the top (tc),
b) on the other hand, the cylindrical internal measurement cavity (Eme) of the measurement block (Cme) is given a slightly truncated conical shape, with an angle at the top (tc)
c) finally, the truncated conical multi-microtubular reaction chamber (Cre) is positioned inside the internal truncated conical measurement cavity (Eme) of the measurement block (Cme) in such a way as to ensure a close contact and to allow:
i) a reduction of the distance between the lateral integral measurement transducer system (T) and the reaction chamber (Cre),
ii) and a possible pressurisation in order to avoid the lateral leakages between the reaction chamber (Cre) and the measurement block (Cme).

10) Method according to claim 6 for the realisation of a sensor for multi-location evaluation of the concentration of analyte elements (aᵢ) of an analyte (A), this method being characterised in that furthermore:
a) on the one hand, the mobile cartridge (Car) including the cylindrical reaction chamber (Cre) is displaced between at least one indication site and a measurement site,
b) on the other hand, the reaction chamber (Cre) is coated with an identifier (Id) prior to the displacement.

11) Method according to claim 1 for the production of a biosensor with sandwich analysis for evaluation of the concentration of analyte elements (aᵢ) of an analyte (A), this method being characterised in that furthermore:
a) a plurality of receptor elements (r1ₘ) of a receptor (R1) (such as an antibody of the analyte (A)) which has an affinity with the analyte elements (aᵢ) in order to detect them is introduced into the interior of the plurality of microtubular channels (cₖ) of the reaction chamber (Cre) and is attached to the internal surfaces thereof (sepₖ),
b) the fraction of the fluid sample (F) is multi-channelled in parallel through the plurality of microtubular channels (cₖ) of the reaction chamber (Cre) in such a way that the analyte elements (aᵢ) come into contact and bond to the receptor elements (r1ₘ) immobilised on the internal surfaces (sepₖ),
c) there are multi-channelled in parallel through the plurality microtubular channels (cₖ) of the reaction chamber (Cre), a plurality of receptor elements (rⱼ) of a receptor (R) (such as an antibody for the analyte (A)) which has an affinity with the analyte elements (aᵢ) in order to detect them and which also has the property [alone or in combination with another active component known as a indicator (U) likewise introduced into the interior of the test volume (Vep)] of modifying by an elementary signal (dE) a measurable extensive state variable (physical and/or chemical) (E), at each occurrence [or according to a certain law of probability], at the time of the chemical bonding between an analyte element (aᵢ) and a receptor element (rⱼ),
d) a lateral integral measurement transducer system (T) for the extensive state variable (E) is positioned
i) entirely outside the enclosing surface (Sev) the reaction chamber (Cre),
ii) and strictly facing the impermeable lateral face (slat),
e) finally, by means of the lateral integral measurement transducer system (T) an integral measurement ΔE=Σ_{k=1 . . . n} Σᵢⱼ (dE)ᵢⱼₖ is carried out (that is to say a summation) of the variations of the said extensive state variable (E), expressing the creation of the complexes (analyte element (aᵢ)=receptor element (rⱼ) and possibly developer (U)) simultaneously for all the elementary volumes (vecₖ) at once, and for all the elementary signals (dE)ᵢⱼₖ in each elementary tube (cₖ) at once through the impermeable lateral face (slat).

12) Method according to claim 1 for the production of a biosensor with displacement analysis for evaluation of the concentration of analyte elements (aᵢ) of an analyte (A), this method being characterised in that furthermore:
a) a plurality of analogue elements (bₘ) of a chemical analogue (B) of the analyte (A) are introduced into the interior of the plurality of microtubular channels (cₖ) of the reaction chamber (Cre) and are attached to the internal surfaces thereof (sepₖ),
b) in the interior of the plurality of microtubular channels (cₖ) of the reaction chamber (Cre) there are multi-channelled in parallel a plurality of receptor elements (rⱼ) of a receptor (R) (such as an antibody Ab common to the analyte (A) and to its analogue (B)) which has an affinity with the analyte elements (aᵢ) and the analogue elements (bₘ) in order to detect them and which also has the property [alone or in combination with another active component known as a indicator (U) likewise introduced into the interior of the test volume (Vep)] of modifying a measurable extensive state variable (physical and/or chemical) (E) by an elementary signal (dE) at each occurrence or according to a certain law of probability at the time of the chemical bonding between an analyte element (aᵢ) or an analogue element (bₘ) and a receptor element (rⱼ), such that the receptor elements (rⱼ) bond to the said analogue elements (bₘ) immobilised on the internal surfaces (sepₖ) of the reaction chamber (Cre),
c) the fraction of the fluid sample (F) is multi-channelled in parallel through the plurality of microtubular channels (cₖ) of the reaction chamber (Cre) in such a way that the analyte elements (aᵢ) competitively bond with the analogue elements (bₘ) and displace some of the receptor elements (rⱼ) immobilised on the internal surfaces (sepₖ) of the reaction chamber (Cre),
d) a lateral integral measurement transducer system (T) for the extensive state variable (E) is positioned
i) entirely outside the enclosing surface (Sev) of the reaction chamber (Cre),
ii) and strictly facing the impermeable lateral face (slat),
e) finally, by means of the lateral integral measurement transducer system (T) an integral measurement ΔE=Σ_{k=1 . . . n} Σᵢⱼₘ (dE)ᵢⱼₖₘ is carried out (that is to say a summation) of the variations of the said extensive state variable (E), each elementary signal (dE)ᵢⱼₖₘ expressing the disappearance of the complexes (analogue element (bₘ)=receptor element (rⱼ)) simultaneously for all the elementary volumes (ve_{ck}) at once, and for all the elementary signals (dE)ᵢⱼₖₘ in each elementary tube (cₖ) at once through the impermeable lateral face (slat).

13) Method according to claim 1 for the production of a biosensor with displacement analysis for evaluation of the concentration of analyte elements (aᵢ) of an analyte (A), this method being characterised in that furthermore:
a) a plurality of receptor elements (rⱼ) of a receptor (R) (such as an antibody Ab of the analyte A) which are immobilised on the internal surfaces (sepₖ) of the reaction chamber (Cre) are introduced into the interior of the plurality of microtubular channels (cₖ) of the reaction chamber (Cre),
b) there are likewise introduced into the interior of the plurality of microtubular channels (cₖ) a plurality
i) of analogue elements (bₘ) of a chemical analogue (B) of the analyte (A),
ii) each conjugated with a indicator element (uₘ) of another active component known as a indicator (U) and capable of modifying a measurable extensive state variable (physical and/or chemical) (E) by an elementary signal (dE),
iii) such that the analogue elements (bₘ) and their conjugated indicator elements (uₘ) are immobilised on the internal surfaces (sepₖ) of the reaction chamber (Cre) in contact with the receptor elements (rⱼ),
c) the fraction of the fluid sample (F) is multi-channelled in parallel through the plurality of microtubular channels (cₖ) in such a way that the analyte elements (aᵢ)
i) enter competitively bond with the analogue elements (bₘ),
ii) take the place of some of the analogue elements (bₘ) and their conjugated indicator elements (uₘ),
iii) and are immobilised on the internal surfaces (sepₖ) of the channels (cₖ) of the reaction chamber (Cre),
d) a lateral integral measurement transducer system (T) for the extensive state variable (E) is positioned
i) entirely outside the enclosing surface (Sev) of the reaction chamber (Cre),
ii) and strictly facing the impermeable lateral face (slat),
e) finally, by means of the lateral integral measurement transducer system (T) an integral measurement ΔE=Σ_{k=1 . . . n} Σᵢⱼₘ (dE)ᵢⱼₖₘ is carried out (that is to say a summation) of the variations of the said extensive state variable (E), each elementary signal (dE)ᵢⱼₖₘ expressing the disappearance of the complexes (analogue element (bₘ)=indicator element (uₘ)) simultaneously for all the elementary volumes (ve_{ck}) at once, and for all the elementary signals (dE)ᵢⱼₖₘ in each elementary tube (cₖ) at once through the impermeable lateral face (slat).

14) Method according to claim 1 for the production of a solid phase biosensor (Sen), the said method previously consisting of:
a) bringing together and disposing substantially parallel a plurality of tubes (C₁, C₂, . . . , Cₖ, . . . , Cₙ) which are initially distant and are made from a fusible material (such as glass),
b) feeding through a treatment furnace (61) the bundle (62) comprising the plurality of tubes (C₁, C₂, . . . , Cₖ, . . . , Cₙ) in such a way as to soften it at a linear feeding speed (Va),
c) downstream of the furnace (62) drawing the bundle (62) comprising the plurality of tubes (C₁, C₂, . . . , Cₖ, . . . , Cₙ) at a drawing speed (Ve) higher than the feeding speed (Va),
d) thus forming by contact and softening a bundle in the form of a continuous monolithic array (65) of microtubular channels (c₁, c₂, . . . , cₖ, . . . , cₙ),
e) periodically dividing this bundle in the form of a continuous array (65) by means of a cutting means (66) in order to form a plurality of monolithic reaction chambers (Cre) in the form of a multi-microtubular array (18),
f) chemically treating each monolithic reaction chamber (Cre) in the form of a multi-microtubular array in such a way as to deposit and fix in a homogeneous manner on the internal surface (sepₖ) of each microtube (cₖ) a plurality of receptor elements (rⱼ) of a receptor component (R) or a plurality of analogue elements (bₘ) of an analogue component (B) [such as an antibody, a nucleic acid, . . . ].

15) Sensor (Sen) for evaluation of the concentration of analyte elements (aᵢ) of an analyte (A) which are present in a fluid sample (F) initially included in a sample volume (Vec), this sensor being of the type constituted by:
a) a reaction chamber (Cre) which provides internally a test volume (Vep)
i) in the interior of which a fraction of the fluid sample (F) is channelled,
ii) circumscribed by an enclosing reaction surface (Sev) constituted by:
a permeable upstream front face (sfam),
a permeable downstream front face (sfav) opposite permeable upstream front face (sfam),
and a substantially cylindrical impermeable lateral face (slat) connected by its two ends to the peripheries of the said two upstream (sfam) and downstream (sfav) faces;
b) at least one active component (chemical and/or biological) known as a receptor (R) which is placed in contact with the fluid sample (F) within the test volume (Vep),
i) of which the receptor elements (rⱼ) have an affinity with the analyte elements (aᵢ) in order to detect them,
ii) and also having the property [alone or in combination with another active component known as a indicator (U) likewise introduced into the interior of the test volume (Vep)] of modifying a measurable extensive state variable (physical and/or chemical) (E) by an elementary signal (dE) at each occurrence [or according to a certain law of probability] at the time of an event of recognition of an analyte element (aᵢ) by a receptor element (rⱼ),
c) a transducer system (T) for measurement of the extensive state variable (E) in order to quantify the presence of the analyte elements (aᵢ) in the fluid sample (F);
this sensor (Sen) being characterised in that in combination:
d) on the one hand, its reaction chamber (Cre) is a multi-microtubular array (18) constituted by the joining of a plurality of cylindrical microtubular channels (cᵢ, c₂, . . . , cₖ, . . . , cₙ) of substantially equal lengths (l) which are disposed substantially parallel and multi-tangent in such a way as to delimit a dense plurality of adjacent separate convex elementary volumes (vec₁, vec₂, . . . , vecₖ, . . . , vecₙ) which are open their two ends (eeₖ, esₖ) and of which the joining forms the non-convex global test volume (Vep), the said non-convex global test volume (Vep) being circumscribed by the enclosing reaction surface (Sev) of which the permeable upstream (sfam) and downstream (sfav) front faces are situated at right angles to the inlet (seₖ) and outlet (ssₖ) sections of the micro-tubular channels (cₖ);
e) on the other hand, its transducer system (T) for lateral integral measurement of the extensive state variable (E),
i) is situated entirely
outside the enclosing surface (Sev) of the reaction chamber (Cre),
and strictly facing the impermeable lateral face (slat),
ii) and delivers an integral measurement ΔE=Σ_{k=1 . . . n} Σᵢⱼ (dE)ᵢⱼₖ, (that is to say a summation) of the variations of the said extensive state variable (E) simultaneously for all the elementary volumes (vecₖ) at once, and for all the elementary signals (dE)ᵢⱼₖ in each microtubular (cₖ) at once through the impermeable lateral face (slat),
in such a way as to quantify globally the presence of the analyte elements (aᵢ) in the fluid sample (F) in all the microtubular channels (Ck) at once.

16) Immunomagnetic sensor (Sen) according to claim 15 of the type in which:
a) a fraction at least of the receptor elements (rⱼ) is combined with indicator elements (uⱼ) of another active component known as a indicator (U) in such a way as to modify a measurable extensive state variable (physical and/or chemical) (E) by an elementary signal (dE) at each occurrence [or according to a certain law of probability], at the time of an event of recognition of an analyte element (aᵢ) by a receptor element (rⱼ),
b) a transducer system (T) for measurement of the extensive state variable (E) in order to quantify the presence of the analyte elements (aᵢ) in the fluid sample (F), comprising
i) an emitter (11) of a magnetic field (H),
ii) and a receiver (13) of a magnetic field (H) connected to a secondary current analysis device (12),
this sensor (Sen) being characterised in that in combination:
c) on the one hand, the indicator elements (uⱼ) are constituted by super-paramagnetic particles [and in particular super-paramagnetic micro-granules (spⱼ)],
d) on the other hand, the said emitter (11) and receiver (13) of a magnetic field (H) are situated
i) entirely outside the enclosing surface (Sev) of the reaction chamber (Cre) in the form of a multi-microtubular array,
ii) and strictly facing the impermeable lateral face (slat).

17) Immunomagnetic sensor (Sen) according to claim 16, characterised in that
a) its magnetic field emitter (11) is formed by a primary winding (71) of coils (74) connected to a primary current source (72),
b) its magnetic field receiver (13) is formed by a secondary winding (73) of coils (74) connected to a secondary current analysis device (12),
c) the primary (71) and secondary (73) windings of coils (74) surround the impermeable lateral face (slat) of the reaction chamber (Cre) in the form of a multi-microtubular array.

18) Sensor (Sen) according to claim 15, characterised in that its reaction chamber (Cre) in the form of a cylindrical monolithic multi-microtubular array (18) is covered with a protective casing (19) in such a way as to form a mobile test cartridge (Car).

19) Sensor (Sen) according to claim 18, characterised in that the protective casing (19) of its reaction chamber (Cre) is cylindrical.

20) Sensor (Sen) according to claim 18, characterised in that the protective casing (19) of its reaction chamber (Cre) is conical.

21) Sensor (Sen) according to claim 18, characterised in that the protective casing (19) of its reaction chamber (Cre) is moulded on over the reaction chamber (Cre).

22) Sensor (Sen) according to claim 18, characterised in that the protective casing (19) of its reaction chamber (Cre) provides internally a reservoir (21) downstream of the reaction chamber (Cre).

23) Sensor (Sen) according to claim 18, characterised in that the protective casing (19) of its reaction chamber (Cre) is equipped with a lateral sealing element.

24) Sensor (Sen) according to claim 23, characterised in that the protective casing (19) of its reaction chamber (Cre) is equipped with a lateral sealing element formed by an annular sealing tongue (20) moulded on at right angles to the upstream end face (22) or the downstream end face (26) of the test cartridge (Car).

25) Sensor (Sen) according to claim 18, characterised in that the protective casing (19) of its reaction chamber (Cre) is provided with an air hole (25) produced on the downstream end face (26) of the cartridge (Cre).

26) Sensor (Sen) according to claim 15, characterised in that a sampling needle (39) is adapted in a sealed and removable manner on the protective casing (19) of the reaction chamber (Cre) facing the upstream face (22) of the cartridge (Cre) situated on the side of the permeable upstream front face (sfam) of the reaction chamber (Cre).

27) Sensor (Sen) according to claim 18, characterised in that the protective casing (19) of the reaction chamber (Cre) is extended:
a) upstream of the upstream end face (22), situated on the side of the permeable upstream front face (sfam) of the multi-microtubular reaction chamber (Cre),
b) in the form of a sampling cone (80) provided with a sampling recess (81) in its end (82).

28) Sensor (Sen) according to claim 18, characterised in that the protective casing (19) of its multi-microtubular reaction chamber (Cre) is covered laterally with an identification label (83).

29) Sensor (Sen) according to claim 15 for evaluation of the concentration of a group of analytes (A₁, A₂, A₃, . . . , Aₚ, . . . ) which are present in a fluid sample (F) initially included in a sample volume (Vec), this sensor being of the type formed by:
a) a multi-stage reactor tube (90), in the interior of which a fraction of the fluid sample (F) is channelled,
b) a plurality of reaction chambers (Cre₁, Cre₂, Cre₃, . . . , Creₚ, . . . ) of substantially equal cross-sections (SCreₚ), disposed coaxially in a chain and sealed laterally within the reactor tube (90),
c) at least a plurality of active components known as receptors (R₁, R₂, R₃, . . . , Rₚ, . . . ) which are placed in contact with the fluid sample (F) within the test volumes (Vepₚ),
i) of which the receptor elements (rₚⱼ) have an affinity with the analyte elements (aₚᵢ) of at least one analyte (Aₚ) in order to detect them,
ii) and also having the property [alone or in combination with another active component known as an indicator (U) likewise introduced into the interior of the test volume (Vep)] of modifying a measurable extensive state variable (physical and/or chemical) (E) by an elementary signal (dE) at each occurrence [or according to a certain law of probability] at the time of an event of recognition of an analyte element (aₚᵢ) by a receptor element (rₚⱼ),
d) a plurality of transducer systems (T₁, T₂, T₃, . . . , Tₚ, . . . ) for lateral integral measurement of the extensive state variable (E), each comprising
i) at least one physical measurement receiver (Rmp₁, Rmp₂, Rmp₃, . . . , Rmpₚ, . . . ) [such as in particular a magnetic field receiver (13)],
ii) each physical measurement receiver (Rmpₚ) fitting over the multi-stage reactor tube (90) at right angles to a corresponding reaction chamber (Creₚ),
e) a system for supplying a plurality of receptor reagents (R₁, R₂, R₃, . . . , Rₚ, . . . ) specific to the analytes (A₁, A₂, A₃, . . . , Aₚ, . . . );
this multi-analyte sensor (Sen) being characterised in that in combination:
f) on the one hand, at least two of the reaction chambers (Creₚ) are formed by a multi-microtubular array (18) formed by the joining of a plurality of cylindrical microtubular channels (cₚ₁, cₚ₂, . . . , cₚₖ, . . . , cₚₙ),
g) on the other hand, at least two transducer systems (T₁, T₂, T₃, . . . , Tₚ, . . . ) for lateral integral measurement of the extensive state variable (E),
i) are situated entirely
outside the enclosing reaction surface (Sevₚ) of the corresponding reaction chamber (Creₚ),
and strictly facing the corresponding impermeable lateral face (slatₚ),
ii) and carry out an integral measurement ΔEₚ=Σ_{k=1 . . . n} Σᵢⱼ (dE)ᵢⱼₚₖ, (that is to say a summation) of the variations of the said extensive state variable (E) simultaneously for all the elementary volumes (vecₚₖ) at once, and for all the elementary signals (dE)ᵢⱼₚₖ in each elementary channel (cₚₖ) at once through the corresponding impermeable lateral face (slatₚ) of the reaction chamber (Creₚ).

30) Sensor (Sen) according to claim 15, further provided with a mobile device (100) for sampling by mobile test cartridge (Car) of analyte elements (aᵢ) of an analyte (A) [that is to say soluble chemical entities or live or dead micro-organisms, or parts of micro-organisms] which are present in a fluid sample (F), comprising the combination between:
a) a sampling block (102) having an internal sampling cavity (103) of a revolutionary shape (cylinder or truncated cone),
b) a mobile cartridge (Car)
i) of a revolutionary shape (cylinder or truncated cone) complementary to that of the internal sampling cavity (103),
ii) including internally a reaction chamber (Cre) in the form of a monolithic multi-microtubular array (18),
iii) introduced in a mobile manner into the internal sampling cavity (103) of the sampling block (102),
iv) and sealed laterally relative to the wall (104) of the internal sampling cavity (103),
c) a means for retaining (105) the mobile cartridge (Car) in the sampling block (102),
d) a means for sealing (106) the sampling block (102) after introduction of the mobile cartridge (Car) into the interior,
i) the said sealing means (106) being possibly merged with the said retaining means (105),
ii) providing after activation at least two openings (111, 112) in the sampling block (102):
an upstream opening (111) for taking the fluid sample (F),
and a downstream opening (112)
e) a pump (115) for movement of the reagents and fluid sample (F), connected to one or the other of the upstream (111) or downstream (112) sampling openings.

31) Sensor (Sen) according to claim 30, characterised in that it further comprises at least one reservoir (122) for chemical and/or biological reagent (and in particular for receptor (R)) connected to one or the other of the:
a) upstream sampling opening (111),
b) and/or downstream sampling opening (112),
of the sampling block (102),
c) and of which the pump (115) for movement of the fluids is situated between the opening (111 or 112) and the reservoir (122) for reagents.

32) Sensor (Sen) according to claim 15, further provided with an independent device for indication after sampling (131) by mobile test cartridge (Car) of analyte elements (aᵢ) of an analyte (A) which are present in a fluid sample (F), comprising the combination between:
a) an indication block having an internal indication cavity of revolutionary shape (cylinder or truncated cone),
b) a mobile cartridge (Car)
i) of a revolutionary shape (cylinder or truncated cone) complementary to that of the internal indication cavity,
ii) including internally a reaction chamber (Cre) in the form of a multi-microtubular array,
iii) introduced in a mobile manner into the internal indication cavity of the indication block,
iv) and sealed laterally relative to the wall of the internal indication cavity,
c) a means for retaining the mobile cartridge (Car) in the indication block,
d) a means for sealing the indication block after introduction of the mobile cartridge (Car) into the interior of the internal indication cavity,
i) the said sealing means possibly being merged with the said retaining means,
ii) providing after activation at least two openings in the indication block:
an upstream opening for introduction of reagents,
and a downstream opening,
e) a pump for movement of the reagents and fluid sample, connected to one or the other of the upstream opening for introduction of reagent or the downstream opening.

33) Sensor (Sen) according to claim 15, further provided with an independent device for measurement after sampling (151) by mobile test cartridge (Car) of analyte elements (aᵢ) of an analyte (A) which are present in a fluid sample (F), comprising the combination between:
a) a transduction block (Cme) providing an internal measurement cavity (Eme) of a revolutionary shape (cylinder or truncated cone),
b) a mobile cartridge (Car)
i) of revolutionary shape (cylinder or truncated cone) complementary to that of the internal measurement cavity (Eme),
ii) including internally a reaction chamber (Cre) in the form of a multi-microtubular array (18),
iii) introduced in a mobile manner into the internal measurement cavity (Eme) of the transduction block (Cme),
iv) and sealed laterally relative to the wall (154) of the internal measurement cavity (Eme),
c) a means for retaining (155) the mobile cartridge (Car) in the transduction block (Cme),
d) a transducer system (T) for lateral integral measurement of the extensive state variable (E), comprising at least one physical measurement receiver [such as in particular a magnetic field receiver (13)], connected to the transduction block (Cme) and situated entirely
i) outside the internal measurement cavity (Eme),
ii) and strictly facing the internal measurement cavity (Eme).

34) Sensor (Sen) according to claim 33, further comprising in combination:
a) a means for sealing (156) the block after introduction of the mobile cartridge (Car) into the interior,
i) the said sealing means (156) being possibly merged with the said retaining means (155),
ii) providing after activation at least two openings in the sampling block:
an upstream feeding opening (161),
and a downstream opening (162),
b) a pump (165) for movement of the sample fluids and/or reagents, connected to one or the other of the upstream (161) or downstream (162) sampling openings.

35) Sensor (Sen) according to claim 15, further provided with a sequential robot device (171) for analysis by mobile test cartridges (Car), formed in combination by:
a) a rigid cartridge support (172) comprising a plurality of blocks (173ₐ, 173_{b}, 173_{c}, 173_{d}, . . . ) spaced from one another at a constant pitch (p),
b) a means for periodic displacement of the cartridge support (172) by a spacing (p′) equal to the said constant pitch (p) such that the plurality of blocks (173ₐ, 173_{b}, 173_{c}, 173_{d}, . . . ) is periodically simultaneously displaced facing an identical plurality of stopping points (181ₐ, 181_{b}, 181_{c}, . . . ),
c) a plurality of mobile test cartridges (Carₐ, Car_{b}, Car_{c}, Car_{d}, . . . )
i) including internally a reaction chamber (Creₐ, Cre_{b}, Cre_{c}, Cre_{d}, . . . ) in the form of a multi-microtubular array (18),
ii) and inserted into the interior of the multitude of blocks (173ₐ, 173_{b}, 173_{c}, 173_{d}, . . . )
d) at least one device for injection of liquid (201ₐ, 201_{b}, 201_{c}, . . . ) (sample and/or reagent) situated facing a stopping point (181ₐ, 181_{b}, 181_{c}, . . . ).

36) Sensor (Sen) according to claim 35, this device (171) being characterised in that furthermore:
a) its rigid cartridge support (172) is formed by a carousel (182),
b) the plurality of blocks (173ₐ, 173_{b}, 173_{c}, 173_{d}, . . . ) is positioned on the periphery of the carousel (182) and they are separated by an equal angle at the top (α),
c) the means for periodic displacement ensures the periodic rotation of the carousel (182) at an angle (α).

37) Sensor (Sen) according to claim 35, this device (171) being characterised in that it further includes a transducer system (T) for lateral integral measurement of the extensive state variable (E), comprising at least one physical measurement receiver (such as in particular a magnetic field receiver (13)),
a) positioned at a stopping point (181ₐ, 181_{b}, 181_{c}, . . . ),
b) periodically mobile perpendicular to the movement of the cartridge support (172),
c) and periodically coming to mount the test cartridge (Car_{d}) situated facing it at the stopping point (181ₐ, 181_{b}, 181_{c}, . . . ), closely surrounding the external surface of the test cartridge (Car_{d}).

38) cartridge (Car₁) for carrying out the method according to claim 1, comprising the characteristic combination between:
a) at least one reaction chamber (Cre₁) in the form of a multi-microtubular array (18),
b) and the protective casing (19) covering the said reaction chamber (Cre₁).

39) Multi-chamber test cartridge (Carm) according to claim 38, characterised in that it further comprises:
a) at least two reaction chambers (Cre₁, . . . , Cre₃, . . . ) in the form of a multi-microtubular array, of identical cross-section, positioned along the axis (zz′),
b) and a protective casing (19) simultaneously covering the reaction chambers (Cre₁, . . . , Cre₃, . . . ).

40) Multi-chamber multi-test cartridge (MCarm) according to claim 38, characterised in that furthermore it is formed by a plurality of test cartridges (Car₁, Car₂, Car₃, . . . ) disposed end to end in series along one and the same axis (zz′).

41) Multi-chamber multi-test cartridge (MCarm) according to claim 40, characterised in that at least two of its test cartridges (Car₁, Car₂, Car₃, . . . ) are fitted into one another.
